(19) United States
(12) Patent Application Publication
(10) Pub. No.: US 2008/0050361 A1
(43) Pub. Date: Feb. 28, 2008

(54) STAPHYLOCOCCUS AUREAS SPECIFIC ANTI-INFECTIVES

(76) Inventors: David E. Heinrichs, London (CA); Jason C. Grigg, Vancouver (CA); Christie Vermeiren, London (CA); Michael E. P. Murphy, Vancouver (CA)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/740,128

(22) Filed: Apr. 25, 2007

Related U.S. Application Data
(63) Continuation-in-part of application No. PCT/IB05/04126, filed on Oct. 25, 2005.
(60) Provisional application No. 60/621,921, filed on Oct. 25, 2004.
(60) Provisional application No. 60/863,550, filed on Oct. 30, 2006.

(51) Int. Cl. A61K 31/395; A61K 31/70; A61K 38/00; A61P 43/00; C12Q 1/00; C12Q 1/68

(52) U.S. Cl. 424/130.1; 435/29; 435/4; 435/6; 514/12; 514/44

(57) ABSTRACT

The present invention provides novel anti-infectives that act on Staphylococcus aureus (S. aureus) iron-regulated surface determinants, IsdA, IsdB, IsdC, IsdH (or HarA).

### CROSS-REFERENCE TO RELATED APPLICATIONS

This application is a continuation-in-part application of International Application No. PCT/IB05/004126, filed Oct. 25, 2005, which claims priority to U.S. Provisional Application No. 60/621,921, filed on Oct. 25, 2004. This application also claims priority to U.S. Provisional Application No. 60/863,550, filed on Oct. 30, 2006. The contents of all of these applications are expressly incorporated herein by reference in their entirety.

### BACKGROUND

Iron is an essential nutrient for almost all organisms and in bacterial infection it is sequestered by host defence mechanisms to limit bacterial growth (Ratledge and Dover, 2000). To combat host iron restriction bacterial pathogens have evolved multiple acquisition systems to obtain iron directly from host sources (Wandersman and Delepelaire, 2004). Heme is the most prevalent form of iron in the human body, representing nearly 75% of the total iron (Stojiljkovic and Perkins-Balding, 2002). Not surprisingly, heme uptake systems are identified as major virulence or colonization factors in bacterial infections (Ahn et al., 2005; Murphy et al., 2002; Stojiljkovic et al., 1995). A recent study eloquently demonstrated that heme is the preferred iron source in the early stages of growth by the versatile pathogen, Staphylococcus aureus (Skaar et al., 2004).

In comparison to Gram-negative bacteria, the molecular basis of iron and in particular heme uptake in Gram-positive bacteria remains poorly understood. Recently, the Isd (Iron-regulated surface determinant) system was identified as a primary heme acquisition pathway in S. aureus (Mazmanian et al., 2002; Mazmanian et al., 2003). Four cell wall anchored proteins, IsdA, IsdB, IsdC, and IsdH (or HarA), are proposed to act as receptors for heme or heme proteins (Dryla et al., 2003; Skaar and Schneewind, 2004). IsdB, which is anchored to the peptidoglycan by the action of sortase A, is exposed on the staphylococcal cell surface (Mazmanian et al., 2003), it is highly immunogenic in mice, and anti-IsdB antibodies cross react with IsdH/HarA (Kuklin et al., 2006). IsdB and IsdH/HarA bind hemoglobin and the hemoglobin-haptoglobin complex, respectively (Dryla et al., 2003; Mazmanian et al., 2003). IsdC, which is anchored to the peptidoglycan by the action of sortase B, is not exposed on the cell surface and therefore likely buried within the thick peptidoglycan structure (Mazmanian et al., 2003). IsdC has been demonstrated to bind free heme (Mack et al., 2004; Mazmanian et al., 2003). IsdA, a sortase A substrate, is expressed highly on the cell surface during iron limited growth (Mazmanian et al., 2003; Taylor and Heinrichs, 2002) and has been demonstrated to bind heme (Mazmanian et al., 2003; Vermeiren et al., 2006). In comparison to wild type S. aureus cells, less heme associates with both whole cells and protoplasts in an isdA null mutant (Mazmanian et al., 2003). In addition to heme binding, other proposed roles for IsdA are in transferrin binding (Taylor and Heinrichs, 2002) and as a broad spectrum adhesin (Clarke et al., 2004). Given that it is at least partly exposed on the bacterial cell surface (Mazmanian et al., 2003), IsdA is the target of a significant titre of IgG antibodies in infected humans and a rat nasal carriage model showed that IsdA has promise as a vaccine candidate (Clarke et al., 2006).

The amino acid sequences of IsdA, IsdB, IsdC and IsdH/HarA contain conserved NEAT domains. These domains are approximately 125 amino acids in length and are named because of the chromosomal location NEAr iron Transport protein-encoding genes (Andrade et al., 2002).

S. aureus is a prevalent human pathogen that causes a wide range of infections ranging from minor skin and wound infections to more serious sequelae such as endocarditis, osteomyelitis and septicemia (Archer (1998) Clin. Infect. Dis. 26:1179-1181). The ability of S. aureus to invade and colonize many tissues may be ascribed to its capacity to express several virulence factors such as fibronectin-, elastin- and collagen-binding proteins that aid in tissue adherence, and multiple exotoxins and proteases that result in tissue destruction and bacterial dissemination. The ability of this bacterium to acquire iron during in vivo growth is also likely important to its pathogenesis, and several research groups have characterized several different genes whose products are involved in the binding and/or transport of host iron compounds (Mazmanian et al., (2003) Science 299:906-9; Modun et al., (1998) Infect. Immun. 66:3591-3596; Taylor and Heinrichs (2002) Mol. Microbiol. 43:1603-1614).

Initially, penicillin could be used to treat even the worst S. aureus infections. However, the emergence of penicillin-resistant strains of S. aureus has reduced the effectiveness of penicillin in treating S. aureus infections and most strains of S. aureus encountered in hospital infections today do not respond to penicillin. Penicillin-resistant strains of S. aureus produce a beta-lactamase, which converts penicillin to pencillinoic acid, and thereby destroys antibiotic activity. Furthermore, the beta-lactamase encoding gene often is propagated episomally, typically on a plasmid, and often is only one of several genes on an episomal element that, together, confer multidrug resistance.

Methicillins, introduced in the 1960s, largely overcame the problem of penicillin resistance in S. aureus. These compounds conserve the portions of penicillin responsible for antibiotic activity and modify or alter other portions that make penicillin a good substrate for inactivating lactamases. However, methicillin resistance has emerged in S. aureus, along with resistance to many other antibiotics effective against this organism, including aminoglycosides, tetracycline, chloramphenicol, macrolides and lincosamides. In fact, methicillin-resistant strains of S. aureus generally are multiply drug resistant. Methicillin-resistant S. aureus (MRSA) has become one of the most important nosocomial pathogens worldwide and poses serious infection control problems. Today, many strains are multiresistant against virtually all antibiotics with the exception of vancomycin-type glycopeptide antibiotics. Drug resistance of S. aureus infections poses significant treatment difficulties, which are likely to get much worse unless new therapeutic agents are developed. Thus, there is an urgent unmet medical need for new and effective therapeutic agents to treat S. aureus infections.

### SUMMARY OF THE INVENTION

The present invention is based, at least in part, on the identification and characterization of Isd (iron-regulated surface determinant) proteins, IsdA, IsdB, IsdC, IsdH (or HarA) which are part of the Isd system involved in the internalization of iron from heme and hemoproteins in S. aureus. IsdA, IsdB, IsdC, IsdH (or HarA) are expressed on the cell surface of S. aureus and are important for iron-restricted growth and survival in vivo. As a result, IsdA, IsdB, IsdC, IsdH (or HarA) proteins and particular domains therein (e.g. NEAT domains) are attractive vaccine targets whose inhibition may lead to compromised bacterial growth in vivo. Further, IsdA, IsdB, IsdC and IsdH (or HarA) proteins and particular domains therein (e.g. NEAT domains) are attractive drug targets that can be used in screening assays to identify S. aureus specific antibiotics.

In one aspect, the invention features Isd protein-based vaccines. In an exemplary embodiment, an Isd based vaccine comprises an IsdA polypeptide and a pharmaceutically acceptable carrier.

In another aspect, the invention features novel antibiotics including antibodies, antisense nucleic acids, and siRNAs that inhibit iron uptake in Stapylococcus aureus (S. aureus). The invention features antibodies against IsdA, IsdB, IsdC and/or IsdH (or HarA) and particular domains therein (e.g. NEAT domains).

In a further aspect, the invention features screening assays for identifying agents that inhibit or otherwise interfere with the expression level and/or function of an Isd protein. In an exemplary embodiment, the invention features screening assays for agents that inhibit the expression and/or function of IsdA. In one embodiment, the assay is a binding assay and an agent that binds to an isd gene product and thereby interferes with its biochemical function is a candidate S. aureus specific antibiotic. In another embodiment, the assay is an expression assay and an agent that reduces the expression level of an Isd polypeptide is a candidate S. aureus specific antibiotic.

In a further aspect, Isd proteins may be expressed on Gram-positive bacteria, including but not limited to, S. aureus, Corynebacterium diphtheriae, Listeria monocytogenes, and Bacillus anthracis. Thus, vaccines and inhibitors that target Isd proteins, as described herein, may be used to treat numerous virulent Gram-positive bacterial strains that cause disease in mammals.

Further features and advantages of the instant disclosed inventions will now be discussed in conjunction with the following Detailed Description and Claims.

### BRIEF DESCRIPTION OF THE DRAWINGS

FIG. 1 shows (A) the nucleic acid sequence encoding IsdA (SEQ ID NO: 1), (B) the reverse complement of SEQ ID NO: 1 (SEQ ID NO: 2), and (C) the corresponding amino acid sequence for IsdA (SEQ ID NO: 3).

FIG. 2 shows (A) the nucleic acid sequence encoding IsdB (SEQ ID NO: 4), (B) the reverse complement of SEQ ID NO: 4 (SEQ ID NO: 5), and (C) the corresponding amino acid sequence for IsdB (SEQ ID NO: 6).

FIG. 3 shows (A) the nucleic acid sequence encoding IsdC (SEQ ID NO: 7), (B) the reverse complement of SEQ ID NO: 7 (SEQ ID NO: 8), and (C) the corresponding amino acid sequence for IsdC (SEQ ID NO: 9).

FIG. 4 is an SDS-PAGE gel that shows whole cell lysates from S. aureus grown in iron-rich media and iron-depleted media.

FIG. 5 is a graph showing S. aureus counts recovered from kidneys of mice 6 days following injection 10⁷ bacteria into the tail vein.

FIG. 6 is a table showing that wild-type S. aureus Isd proteins bound to heme can survive under conditions of increased hydrogen peroxide (H₂O₂) compared to S. aureus strains where isdA, isdB, isdC were knocked-out (✓, indicates greater than 90% survival of bacteria).

FIGS. 7A and 7B are SDS-PAGE gels showing proteins from wild type and isdA knockout S. aureus (A) stained with coomassie (for total protein) and (b) stained with TMBZ (tetramethylbenzidine).

FIGS. 8A and 8B are gels showing the peroxidase activity in S. aureus cell wall extracts. Cell wall proteins from S. aureus Newman (lanes 1 and 2), S. aureus Newman isdA mutant (lanes 3 and 4) and S. aureus Newman isdA mutant complemented with pJT35 (lanes 5 and 6) were incubated with (lanes 2, 4, 6) or without heme (lanes 1, 3, 5) prior to electrophoresis. Gels were then stained with coomassie brilliant blue R-250 (panel A) or 3, 3′, 5, 5′ tetramethyl benzidine (TMBZ) (panel B). The TMBZ stained bands indicate the presence of heme-dependent peroxidase activity.

FIGS. 9A and 9B are graphs showing that IsdA enhances S. aureus growth on hemin as a sole source of iron. (A) Plate bioassays were used to measure growth on hemin as a sole source of iron for S. aureus Newman, H734 (isdA::tet), and H734 containing a multicopy plasmid expressing isdA. The asterisk indicates a statistically significant change in growth promotion compared to Newman or H734 (P<0.001 as determined by Student t-test). Solid horizontal line denotes the diameter of the paper disk that contained the hemin. (B) Liquid culture assays were used to compare the growth of S. aureus strains Newman (squares), H734 (circles) and H734 containing pJT35 (triangles) in TMS media containing 10 μM EDDHA with 50 μM FeCl₃ (gray fill), 5 μg/mL hemin (black fill) or no further additions (no fill). Data points represent the mean of 3 replicates and error bars represent standard deviations.

FIG. 10 shows the overall structure of the IsdA NEAT domain heme complex Heme carbon, nitrogen and iron atoms are shown in red, blue, and orange, respectively.

FIG. 11A shows the secondary structure of the NEAT domain with the heme and selected amino acid residues drawn as sticks. Oxygen and nitrogen atoms are red and blue, respectively. The heme carbon atoms are in red and the carbons of amino acid side chains shown in yellow. FIG. 11B shows the electrostatic potential surface representation of the domain in the same orientations as in FIG. 11A. Positive potentials are indicated in blue and negative potentials are in red. Heme (green) is in the binding pocket. FIG. 11C shows a superposition of the backbone of the apo (yellow) and holo (magenta) structures of the IsdA NEAT domain. Heme is shown in sticks within the binding pocket. In FIG. 11D, the superposition of residues in the heme binding pocket of both the apo (yellow) and holo (magenta) protein are drawn in sticks and are labelled. Nitrogen (blue), oxygen and sulphur (orange) atoms are indicated by colour in displayed side-chains.

FIG. 12 is a stereo view of the heme site including the residues of the heme binding pocket. The electron density represented in gray is a 2Fo-Fc map contoured at 1.0 σ. Carbon atoms of the heme and the amino acid residues are shown in red and yellow, respectively. Nitrogen, oxygen and iron atoms are blue, red and magenta, respectively.

FIG. 13 is an electronic spectra of wild-type and alanine-substitution mutants of GST-IsdA fusion proteins expressed and isolated from E. Coli.

FIG. 14 shows a multiple sequence alignment of NEAT domains from S. aureus. The alignment was extracted from an alignment of NEAT domains from several Gram-positive organisms (FIG. S1). In the designation for each of the domains, the number before the dash identifies the NEAT domain number in order from the N-terminus of the protein, and the number after the dash indicates the total number of NEAT domains present in the protein. Positions shaded in dark gray and light gray are identical or similar, respectively, in at least 85% of the aligned S. aureus sequences. Residues identified by asterisk are those that contribute contacts to the heme group. The primary accession numbers are as follows:SauIsdA (Q7A655), SauIsdB (Q7A656), SauIsdC (Q7A654), SauHarA (Q99TD3).

### DETAILED DESCRIPTION OF THE INVENTION

1. General

As described herein, the internalization of iron through the uptake of heme is a virulence property that may be attenuated when isd genes, such as isdA, isdB, isdC IsdH (or HarA) are knocked out. Further, as described herein, heme-bound Isd proteins may serve an additional role in promoting S. aureus survival in the host. Heme-bound Isd proteins appear to serve as an oxidative buffer that protects cells form the detrimental effects of free radicals. Therefore, mutants lacking expression of Isd proteins are more susceptible to challenges with hydrogen peroxide whereas wild type S. aureus can survive in higher concentrations of hydrogen peroxide.

The Isd proteins, as described herein, are essential for S. aureus infection in vivo and are highly expressed during S. aureus infection. As S. aureus enters a host, it encounters an environment that is iron-limited and Isd protein expression is subsequently up regulated. In the iron-limited host, Isd expression likely remains up regulated as the S. aureus scavenge for iron. IsdA, in particular, as described herein is immunodominant, since a 1:4000 dilution of serum from convalescent patients (i.e., patients suffering from S. aureus infections) reacted positively in Western immunoblots with 4 micrograms of purified IsdA protein. Thus, Isd proteins are attractive targets for vaccine development. Antigenic peptides of Isd proteins may be used as vaccine targets to generate an effective immune response against S. aureus. Further, inhibiting the function of Isd proteins using an Isd specific antibody, antisense RNA, siRNA or small molecule inhibitor may be an effective way of attenuating the virulence of S. aureus.

The Isd proteins described herein are expressed on S. aureus. In further embodiments, Isd proteins may be expressed on other Gram-positive bacteria. Non-limiting examples of Gram-positive pathogens expressing Isd proteins include S. aureus, Corynebacterium diphtheriae, Listeria monocytogenes, and Bacillus anthracis. Thus, vaccines and inhibitors that target Isd proteins, as described herein, may be used to treat other virulent Gram-positive bacterial strains that cause disease in mammals.

2. Definitions

For convenience, the meaning of certain terms and phrases employed in the specification, examples, and appended claims are provided below. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

As used herein, the term “adjuvant” refers to a substance that elicits an enhanced immune response when used in combination with a specific antigen.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. Screening assays described herein below may identify agents. Such agents may be inhibitors or antagonists of Isd mediated iron uptake in Staphylococcus aureus. The activity of such agents may render it suitable as a “therapeutic agent” which is a biologically, physiologically, or pharmacologically active substance (or substances) that acts locally or systemically in a subject.

The terms “antagonist” or “inhibitor” refer to an agent that down regulates (e.g., suppresses or inhibits) at least one bioactivity of a protein. An antagonist may be a compound which inhibits or decreases the interaction between a protein and another molecule, e.g., a target peptide or enzyme substrate. An antagonist may also be a compound that down regulates expression of a gene or which reduces the amount of expressed protein present.

As used herein the term “antibody” refers to an immunoglobulin and any antigen-binding portion of an immunoglobulin (e.g., IgG, IgD, IgA, IgM and IgE) i.e. a polypeptide that contains an antigen-binding site, which specifically binds (“immunoreacts with”) an antigen. Antibodies can comprise at least one heavy (H) chain and at least one light (L) chain interconnected by at least one disulfide bond. The term “V_{H}” refers to a heavy chain variable region of an antibody. The term “V_{L}” refers to a light chain variable region of an antibody. In exemplary embodiments, the term “antibody” specifically covers monoclonal and polyclonal antibodies. A “polyclonal antibody” refers to an antibody, which has been derived from the sera of animals immunized with an antigen or antigens. A “monoclonal antibody” refers to an antibody produced by a single clone of hybridoma cells. Techniques for generating monoclonal antibodies include, but are not limited to, the hybridoma technique (see Kohler & Milstein (1975) Nature 256:495-497); the trioma technique; the human B-cell hybridoma technique (see Kozbor, et al. (1983) Immunol. Today 4:72), the EBV hybridoma technique (see Cole, et al., 1985 In: Monoclonal Antibodies and Cancer Therapy, Alan R. Liss, Inc., pp. 77-96) and phage display.

Polyclonal or monoclonal antibodies can be further manipulated or modified to generate chimeric or humanized antibodies. “Chimeric antibodies” are encoded by immunoglobulin genes that have been genetically engineered so that the light and heavy chain genes are composed of immunoglobulin gene segments belonging to different species. For example, substantial portions of the variable (V) segments of the genes from a mouse monoclonal antibody, e.g., obtained as described herein, may be joined to substantial portions of human constant (C) segments. Such a chimeric antibody is likely to be less antigenic to a human than a mouse monoclonal antibody

As used herein, the term “humanized antibody” (HuAb) refers to a chimeric antibody with a framework region substantially identical (i.e., at least 85%) to a human framework, having CDRs from a non-human antibody, and in which any constant region has at least about 85-90%, and preferably about 95% polypeptide sequence identity to a human immunoglobuhn constant region. See, for example, PCT Publication WO 90/07861 and European Patent No. 0451216. All parts of such a HuAb, except possibly the CDRs, are substantially identical to corresponding parts of one or more native human immunoglobulin sequences. The term “framework region” as used herein, refers to those portions of immunoglobulin light and heavy chain variable regions that are relatively conserved (i.e., other than the CDRs) among different immunoglobulins in a single species, as defined by Kabat, et al. (1987) Sequences of Proteins of Immunologic Interest, 4^{th} Ed., US Dept. Health and Human Services. Human constant region DNA sequences can be isolated in accordance with well known procedures from a variety of human cells, but preferably from immortalized B cells. The variable regions or CDRs for producing humanized antibodies may be derived from monoclonal antibodies capable of binding to the antigen, and will be produced in any convenient mammalian source, including mice, rats, rabbits, or other vertebrates.

The term “antibody” also encompasses antibody fragments. Examples of antibody fragments include Fab, Fab′, Fab′-SH, F(ab′)₂, and Fv fragments; diabodies and any antibody fragment that has a primary structure consisting of one uninterrupted sequence of contiguous amino acid residues, including without limitation: single-chain Fv (scFv) molecules, single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s). Suitable leucine zipper sequences include the jun and fos leucine zippers taught by Kostelney et al., (1992) J. Immunol., 148: 1547-1553 and the GCN4 leucine zipper described in U.S. Pat. No. 6,468,532. Fab and F(ab′)₂ fragments lack the Fe fragment of intact antibody and are typically produced by proteolytic cleavage, using enzymes such as papain (to produce Fab fragments) or pepsin (to produce F(ab′)₂ fragments).

An antibody “specifically binds” to an antigen or an epitope of an antigen if the antibody binds preferably to the antigen over most other antigens. For example, the antibody may have less than about 50%, 20%, 10%, 5%, 1% or 0.1% cross-reactivity toward one or more other epitopes.

The term “conservative substitutions” refers to changes between amino acids of broadly similar molecular properties. For example, interchanges within the aliphatic group alanine, vahne, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Preferred conservative substitution groups are aspartate-glutamate; asparagine-glutamine; valine-leucine-isoleucine; alanine-valine; valine-leucine-isoleucine-methionine; phenylalanine-tyrosine; phenylalanine-tyrosine-tryptophan; lysine-arginine; and histidine-lysine-arginine.

An “effective amount” is an amount sufficient to produce a beneficial or desired clinical result upon treatment. An effective amount can be administered to a patient in one or more doses. In terms of treatment, an effective amount is an amount that is sufficient to decrease an infection in a patient. Several factors are typically taken into account when determining an appropriate dosage to achieve an effective amount. These factors include age, sex and weight of the patient, the condition being treated, the severity of the condition and the form and effective concentration of the agent administered.

The term “epitope” refers to that region of an antigen to which an antibody binds preferentially and specifically. A monoclonal antibody binds preferentially to a single specific epitope of a molecule that can be molecularly defined. An epitope of a particular protein maybe constituted by a limited number of amino acid residues, e.g. 5-15 residues that are either in a linear or non-linear organization on the protein.

“Equivalent” when used to describe nucleic acids or nucleotide sequences refers to nucleotide sequences encoding functionally equivalent polypeptides. Equivalent nucleotide sequences will include sequences that differ by one or more nucleotide substitution, addition or deletion, such as an allelic variant; and will, therefore, include sequences that differ due to the degeneracy of the genetic code. For example, nucleic acid variants may include those produced by nucleotide substitutions, deletions, or additions. The substitutions, deletions, or additions may involve one or more nucleotides. The variants may be altered in coding regions, non-coding regions, or both. Alterations in the coding regions may produce conservative or non-conservative amino acid substitutions, deletions or additions.

“Homology” or alternatively “identity” refers to sequence similarity between two peptides or between two nucleic acid molecules. Homology may be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. The term “percent identical” refers to sequence identity between two amino acid sequences or between two nucleotide sequences. Identity may be determined by comparing a position in each sequence, which may be aligned for purposes of comparison. When an equivalent position in the compared sequences is occupied by the same base or amino acid, then the molecules are identical at that position; when the equivalent site is occupied by the same or a similar amino acid residue (e.g., similar in steric and/or electronic nature), then the molecules may be referred to as homologous (similar) at that position. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and may be used with, e.g., default settings. ENTREZ is available through the National Center for Biotechnology Information, National Library of Medicine, National Institutes of Health, Bethesda, Md. In one embodiment, the percent identity of two sequences may be determined by the GCG program with a gap weight of 1, e.g., each amino acid gap is weighted as if it were a single amino acid or nucleotide mismatch between the two sequences. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif. USA. Preferably, an alignment program that permits gaps in the sequence is utilized to align the sequences. The Smith-Waterman is one type of algorithm that permits gaps in sequence alignments. See Meth. Mol. Biol 70: 173-187 (1997). Also, the GAP program using the Needleman and Wunsch alignment method may be utilized to align sequences. An alternative search strategy uses MPSRCH software, which runs on a MASPAR computer. MPSRCH uses a Smith-Waterman algorithm to score sequences on a massively parallel computer. This approach improves the ability to pick up distantly related matches, and is especially tolerant of small gaps and nucleotide sequence errors. Nucleic acid-encoded amino acid sequences may be used to search both protein and DNA databases. Databases with individual sequences are described in Methods in Enzymology, ed. Doolittle, supra. Databases include Genbank, EMBL, and DNA Database of Japan (DDBJ).

As used herein, the term “infection” refers to an invasion and the multiplication of microorganisms such as S. aureus in body tissues, which may be clinically unapparent or result in local cellular injury due to competitive metabolism, toxins, intracellular replication or antigen antibody response. The infection may remain localized, subclinical and temporary if the body's defensive mechanisms are effective. A local infection may persist and spread by extension to become an acute, subacute or chronic clinical infection or disease state. A local infection may also become systemic when the microorganisms gain access to the lymphatic or vascular system.

The terms “iron-regulated surface determinant system” or “Isd system” as used herein, refers to the S. aureus Isd locus, which comprises numerous genes encoded by five different transcriptional units that, together, encode a putative heme uptake system. The five transcriptional units are isdA, isdB, isdCDEFsrtBisdG, isdH, and isdI. Transcription of isd genes is regulated by environment iron on the control of the Fur promoter. Four of the proteins encoded by the Isd locus, IsdA, IsdB, IsdC, and IsdH, are covalently anchored to the cell wall by an amide linkage between the C-terminal end of the polypeptide chain and peptidoglycan. IsdA, IsdB, and IsdH are characterized as having a C-terminal sorting signal referred to as an LPXTG motif (i.e., a motif recognized by sortase A). IsdC is characterized as having a C-terminal sorting signal referred to as an NPQTN motif (i.e., a motif recognized by sortase B in S. aureus). IsdA, IsdB, and IsdH are anchored to the cell wall by a sortase A (srtA), a membrane anchored transpeptidase that cleaves cell surface proteins at the LPXTG motif and catalyzes the formation of the amide bond between the polypeptide and peptidoglycan IsdC is anchored to the cell wall by sortase B (srtB), a transpeptidase similar to sortase A. Other proteins encoded by the Isd locus, include IsdD, IsdE, and IsdF, which are putative membrane translocation factors, and IsdG and IsdI, which are cytoplasmic heme-iron binding proteins, that may be involved in extracting iron from heme.

“IsdA polypeptide” as used herein refers to iron-regulated surface determinant A. The full-length sequence of the IsdA polypeptide is as set forth in SEQ ID NO: 3 and is encoded by SEQ ID NO: 1. The term also encompasses any fragments (including, for example, the NEAT domain), variants, analogs, agonists, chemical derivatives, functional derivatives or functional fragments of an IsdA polypeptide. “IsdA immunogens” are IsdA polypeptides, which are capable of eliciting an immune response in a subject.

“IsdB polypeptide” as used herein refers to iron-regulated surface determinant B. The full-length sequence of the IsdB polypeptide is as set forth in SEQ ID NO: 6 and is encoded by SEQ ID NO: 4. The term also encompasses any fragments (including, for example, the NEAT domain), variants, analogs, agonists, chemical derivatives, functional derivatives or functional fragments of an IsdB polypeptide. “IsdB immunogens” are IsdB polypeptides, which are capable of eliciting an immune response in a subject.

“IsdC polypeptide” as used herein refers to iron-regulated surface determinant C. The full-length sequence of IsdC polypeptide is as set forth in SEQ ID NO: 9 and is encoded by SEQ ID NO: 7. The term also encompasses any fragments (including, for example, the NEAT domain), variants, analogs, agonists, chemical derivatives, functional derivatives or functional fragments of an IsdC polypeptide. “IsdC immunogens” are IsdC polypeptides, which are capable of eliciting an immune response in a subject. IsdH (or HarA)

“Label” and “detectable label” refer to a molecule capable of detection including, but not limited to radioactive isotopes, fluorophores, chemiluminescent moieties, enzymes, enzyme substrates, enzyme cofactors, enzyme inhibitors, dyes, metal ions, ligands (e.g., biotin or haptens) and the like. “Fluorophore” refers to a substance or a portion thereof which is capable of exhibiting fluorescence in the detectable range. Particular examples of appropriate labels include fluorescein, rhodamine, dansyl, umbelliferone, Texas red, luminol, NADPH, alpha- or beta-galactosidase and horseradish peroxidase.

As used herein with respect to genes, the term “mutant” refers to a gene, which encodes a mutant protein. As used herein with respect to proteins, the term “mutant” means a protein, which does not perform its usual or normal physiological role. S. aureus polypeptide mutants may be produced by amino acid substitutions, deletions or additions. The substitutions, deletions, or additions may involve one or more residues. Especially preferred among these are substitutions, additions and deletions, which alter the properties and activities of a S. aureus protein.

The terms “polynucleotide”, and “nucleic acid” are used interchangeably to refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, antisense nucleic acids, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term “recombinant” polynucleotide means a polynucleotide of genomic, cDNA, semisynthetic, or synthetic origin, which either does not occur in nature or is linked to another polynucleotide in a non-natural arrangement. An “oligonucleotide” refers to a single stranded polynucleotide having less than about 100 nucleotides, less than about, e.g., 75, 50, 25, or 10 nucleotides.

The terms “polypeptide”, “peptide” and “protein” (if single chain) are used interchangeably herein to refer to polymers of amino acids. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The term “small molecule” refers to a compound, which has a molecular weight of less than about 5 kD, less than about 2.5 kD, less than about 1.5 kD, or less than about 0.9 kD. Small molecules may be, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides or polypeptides.

The term “specifically hybridizes” refers to detectable and specific nucleic acid binding. Polynucleotides, oligonucleotides and nucleic acids of the invention selectively hybridize to nucleic acid strands under hybridization and wash conditions that minimize appreciable amounts of detectable binding to nonspecific nucleic acids. Stringent conditions may be used to achieve selective hybridization conditions as known in the art and discussed herein. Generally, the nucleic acid sequence homology between the polynucleotides, oligonucleotides, and nucleic acids of the invention and a nucleic acid sequence of interest will be at least 30%, 40%, 50%, 60%, 70%, 80%, 85%, 90%, 95%, 98%, 99%, or more. In certain instances, hybridization and washing conditions are performed under stringent conditions according to conventional hybridization procedures and as described further herein.

The terms “stringent conditions” or “stringent hybridization conditions” refer to conditions, which promote specific hybridization between two complementary polynucleotide strands so as to form a duplex. Stringent conditions may be selected to be about 5° C. lower than the thermal melting point (Tm) for a given polynucleotide duplex at a defined ionic strength and pH. The length of the complementary polynucleotide strands and their GC content will determine the Tm of the duplex, and thus the hybridization conditions necessary for obtaining a desired specificity of hybridization. The Tm is the temperature (under defined ionic strength and pH) at which 50% of a polynucleotide sequence hybridizes to a perfectly matched complementary strand. In certain cases it may be desirable to increase the stringency of the hybridization conditions to be about equal to the Tm for a particular duplex.

A variety of techniques for estimating the Tm are available. Typically, G-C base pairs in a duplex are estimated to contribute about 3° C. to the Tm, while A-T base pairs are estimated to contribute about 2° C., up to a theoretical maximum of about 80-100° C. However, more sophisticated models of Tm are available in which G-C stacking interactions, solvent effects, the desired assay temperature and the like are taken into account. For example, probes can be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the formation of the duplex.

Hybridization may be carried out in 5×SSC, 4×SSC, 3×SSC, 2×SSC, 1×SSC or 0.2×SSC for at least about 1 hour, 2 hours, 5 hours, 12 hours, or 24 hours. The temperature of the hybridization may be increased to adjust the stringency of the reaction, for example, from about 25° C. (room temperature), to about 45° C., 50° C., 55° C., 60° C., or 65° C. The hybridization reaction may also include another agent affecting the stringency, for example, hybridization conducted in the presence of 50% formamide increases the stringency of hybridization at a defined temperature.

The hybridization reaction may be followed by a single wash step, or two or more wash steps, which may be at the same or a different salinity and temperature. For example, the temperature of the wash may be increased to adjust the stringency from about 25° C. (room temperature), to about 45° C., 50° C., 55° C., 60° C., 65° C., or higher. The wash step may be conducted in the presence of a detergent, e.g., 0.1 or 0.2% SDS. For example, hybridization may be followed by two wash steps at 65° C. each for about 20 minutes in 2×SSC, 0.1% SDS, and optionally two additional wash steps at 65° C. each for about 20 minutes in 0.2×SSC, 0.1% SDS.

Exemplary stringent hybridization conditions include overnight hybridization at 65° C. in a solution comprising, or consisting of, 50% formamide, 10× Denhardt (0.2% Ficoll, 0.2% Polyvinylpyrrolidone, 0.2% bovine serum albumin) and 200 μg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization may consist of hybridizing two nucleic acids in solution, or a nucleic acid in solution to a nucleic acid attached to a solid support, e.g., a filter. When one nucleic acid is on a solid support, a prehybridization step may be conducted prior to hybridization Prehybridization may be carried out for at least about 1 hour, 3 hours or 10 hours in the same solution and at the same temperature as the hybridization solution (without the complementarypolynucleotide strand).

Appropriate stringency conditions are known to those skilled in the art or may be determined experimentally by the skilled artisan. See, for example, Current Protocols in Molecular Biology, John Wiley & Sons, N.Y. (1989), 6.3.1-12.3.6; Sambrook et al., 1989, Molecular Cloning, A Laboratory Manual, Cold Spring Harbor Press, N.Y; S. Agrawal (ed.) Methods in Molecular Biology, volume 20; Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York; and Tibanyenda, N. et al., Eur. J. Biochem. 139:19 (1984) and Ebel, S. et al., Biochem. 31:12083 (1992).

The term “substantially homologous” when used in connection with a nucleic acid or amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

A “subject” refers to a male or female mammal, including humans.

A “variant” of an Isd polypeptide refers to a molecule, which is substantially similar to IsdA, IsdB, of IsdC. Variant peptides may be covalently prepared by direct chemical synthesis of the variant peptide, using methods well known in the art. Variants of Isd polypeptides may further include, for example, deletions, insertions or substitutions of residues within the amino acid sequence. Any combination of deletion, insertion, and substitution may also be made to arrive at the final construct, provided that the final construct possesses the desired activity. These variants may be prepared by site-directed mutagenesis, (as exemplified by Adelman et al., DNA 2: 183 (1983)) of the nucleotides in the DNA encoding the peptide molecule thereby producing DNA encoding the variant and thereafter expressing the DNA in recombinant cell culture. The variants typically exhibit the same qualitative biological activity as wild type Isd polypeptides. It is known in the art that one may also synthesize all possible single amino acid substitutions of a known polypeptide (Geysen et al., Proc. Nat. Acad. Sci. (USA) 18:3998-4002 (1984)). While the effects of different substitutions are not always additive, it is reasonable to expect that two favorable or neutral single substitutions at different residue positions in an Isd polypeptide can safely be combined without losing any Isd protein activity. Methods for the preparation of degenerate polypeptides are as described in Rutter, U.S. Pat. No. 5,010,175; Haughter et al., Proc. Nat. Acad. Sci. (USA) 82:5131-5135 (1985); Geysen et al., Proc. Nat. Acad. Sci. (USA) 18:3998-4002 (1984); WO86/06487; and WO86/00991. In devising a substitution strategy, a person of ordinary skill would determine which residues to vary and which amino acids or classes of amino acids are suitable replacements. One may also take into account studies of sequence variations in families or naturally occurring homologous proteins. Certain amino acid substitutions are more often tolerated than others, and these are often correlated with similarities in size, charge, etc., between the original amino acid and its replacement. Insertions or deletions of amino acids may also be made, as described above. The substitutions are preferably conservative, see, e.g., Schulz et al., Principle of Protein Structure (Springer-Verlag, New York (1978)); and Creighton, Proteins: Structure and Molecular Properties (W. H. Freeman & Co., San Francisco (1983)); both of which are hereby incorporated by reference in their entireties.

A “chemical derivative” of an Isd polypeptide can contain additional chemical moieties not normally part of the IsdA, IsdB, or IsdC amino acid sequences. Such chemical modifications may be introduced into an Isd polypeptide by reacting targeted amino acid residues of the polypeptide with an organic derivatizing agent that is capable of reacting with selected side chains or terminal residues. Amino terminal residues can be reacted with succinic or other carboxylic acid anhydrides. Other suitable reagents for derivatizing alpha-amino-containing residues include amido-esters such as methyl picolinimidate; pyridoxal phosphate; pyridoxal; chloroborohydride; trinitrobenzenesulfonic acid; O-methylisourea; 2,4-pentanedione; and transaminase-catalase reacted with glyoxylate. Specific modifications of tyrosyl residues per se have been studied extensively, with particular interest in introducing spectral labels into tyrosyl residues by reaction with aromatic diazonium compounds or tetranitromethane. Most commonly, N-acetylimidazole and tetranitromethane are use to form O-acetyl tyrosyl species and 3-nitro derivatives, respectively. Carboxyl side groups such as aspartyl or glutamyl can be selectively modified by reaction with carbodiimides (R′N—C—N—R′) such as 1-cyclohexy-3-[2-morpholinyl-(4-ethyl)]carbodiimide or 1-ethyl-3-(4-azonia-4,4-dimethylpentyl)carbodiimide. Furthermore, aspartyl and glutamyl residues can be converted to asparaginyl and glutaminyl residues by reaction with ammonium ions.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

3. Isd Genes

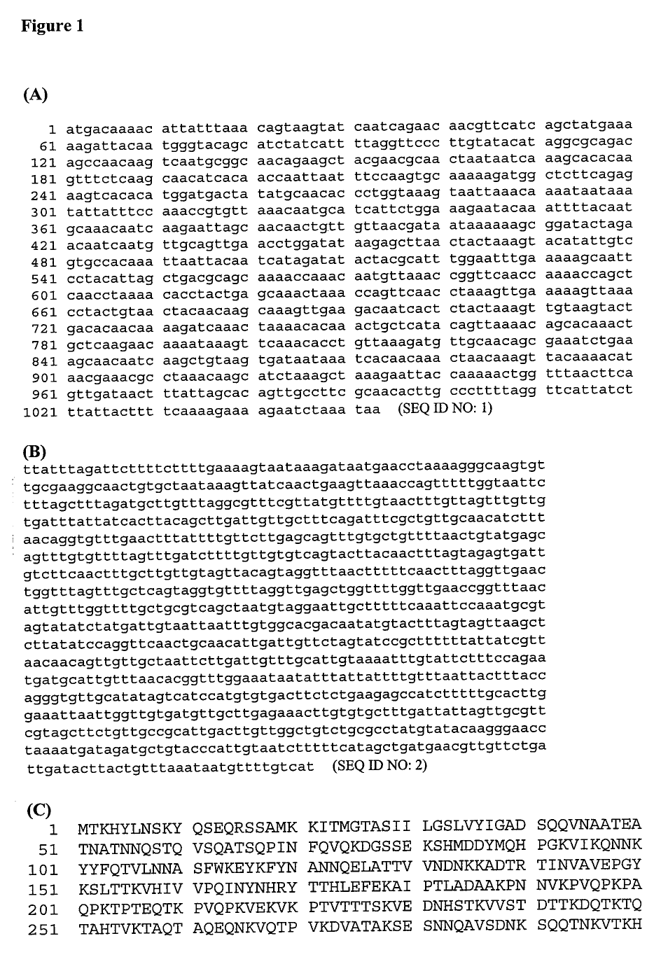

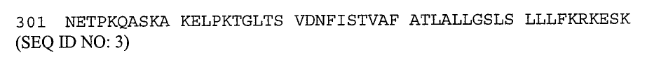

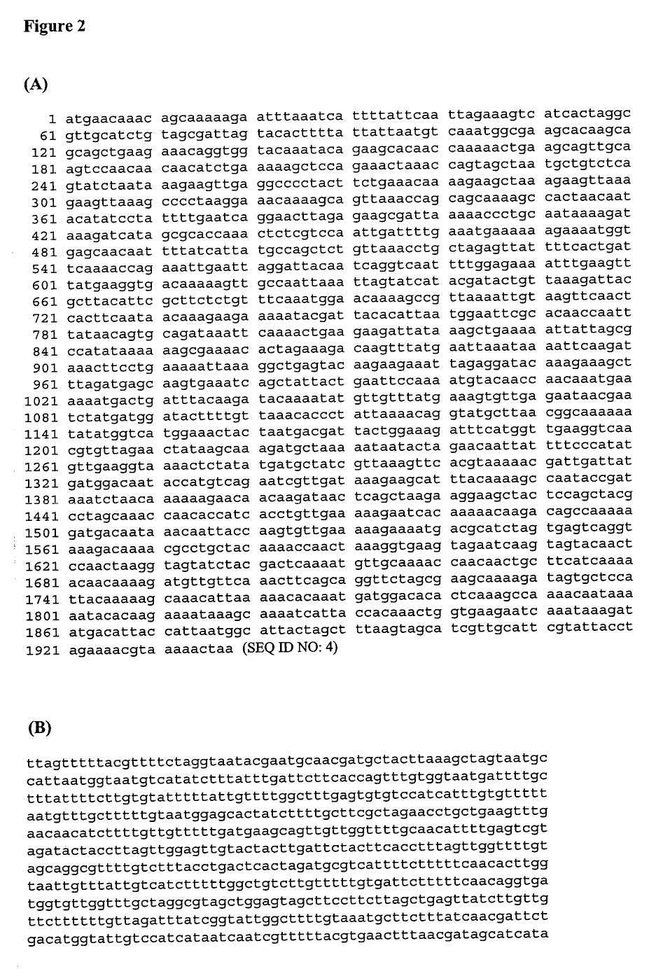

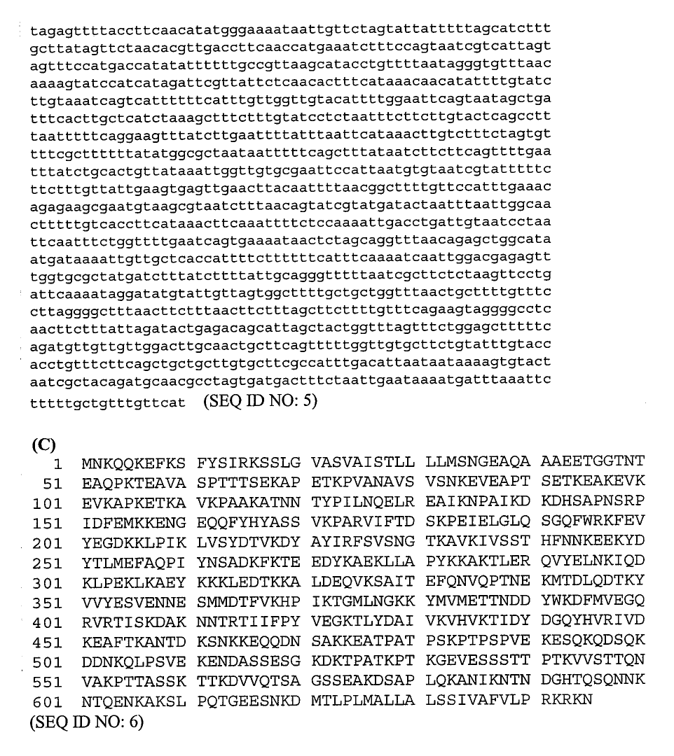

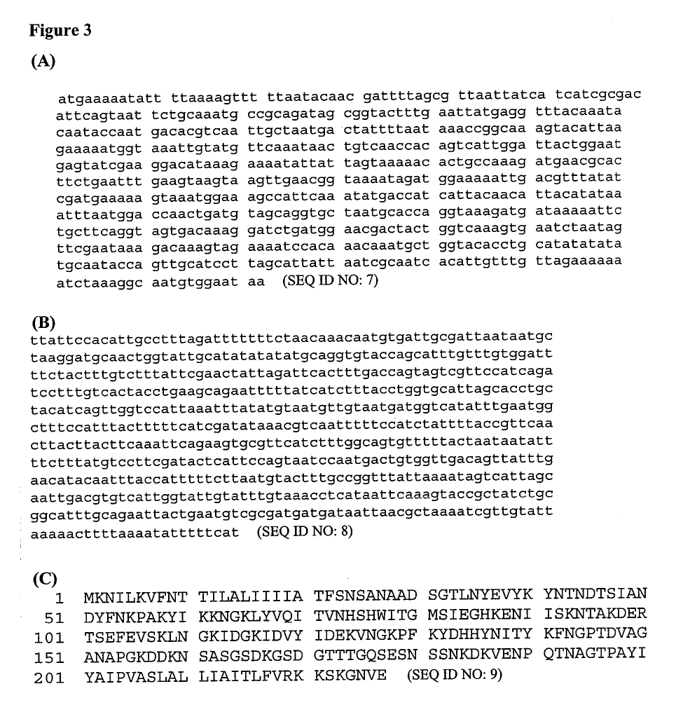

Three genes of the Isd locus, isdA, isdB, and isdC, encode cell surface proteins, which are covalently anchored to the S. aureus cell wall. FIGS. 1-3 provide the nucleic acid sequences of isdA (SEQ ID NO: 1), isdB (SEQ ID NO: 4), and isdC (SEQ ID NO: 7).

Nucleic acids of the present invention may also comprise, consist of or consist essentially of any of the isd nucleotide sequences described herein. Yet other nucleic acids comprise, consist of or consist essentially of a nucleotide sequence that has at least about 70%, 80%, 90%, 95%, 98% or 99% identity or homology with an isd gene. Substantially homologous sequences may be identified using stringent hybridization conditions.

Isolated nucleic acids which differ from the nucleic acids of the invention due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the polypeptides of the invention will exist. One skilled in the art will appreciate that these variations in one or more nucleotides (from less than 1% up to about 3 or 5% or possibly more of the nucleotides) of the nucleic acids encoding a particular protein of the invention may exist among a given species due to natural allelic variation. Any and all such nucleotide variations and resulting amino acid polymorphisms are within the scope of this invention.

Nucleic acids encoding proteins which have amino acid sequences evolutionarily related to a polypeptide disclosed herein are provided, wherein “evolutionarily related to”, refers to proteins having different amino acid sequences which have arisen naturally (e.g., by allelic variance or by differential splicing), as well as mutational variants of the proteins of the invention which are derived, for example, by combinatorial mutagenesis.

Fragments of the polynucleotides of the invention encoding a biologically active portion of the subject polypeptides are also provided. As used herein, a fragment of a nucleic acid encoding an active portion of a polypeptide disclosed herein refers to a nucleotide sequence having fewer nucleotides than the nucleotide sequence encoding the full length amino acid sequence of a polypeptide of the invention, and which encodes a given polypeptide that retains at least a portion of a biological activity of the full-length Isd protein as defined herein, or alternatively, which is functional as a modulator of the biological activity of the full-length protein. For example, such fragments include a polypeptide containing a domain of the full-length protein from which the polypeptide is derived that mediates the interaction of the protein with another molecule (e.g., polypeptide, DNA, RNA, etc.).

Nucleic acids provided herein may also contain linker sequences, modified restriction endonuclease sites and other sequences useful for molecular cloning, expression or purification of such recombinant polypeptides.

A nucleic acid encoding an Isd polypeptide provided herein may be obtained from mRNA or genomic DNA from any organism in accordance with protocols described herein, as well as those generally known to those skilled in the art. A cDNA encoding a polypeptide of the invention, for example, may be obtained by isolating total mRNA from an organism, for example, a bacteria, virus, mammal, etc. Double stranded cDNAs may then be prepared from the total mRNA, and subsequently inserted into a suitable plasmid or bacteriophage vector using any one of a number of known techniques. A gene encoding a polypeptide of the invention may also be cloned using established polymerase chain reaction techniques in accordance with the nucleotide sequence information provided by the invention. In one aspect, methods for amplification of a nucleic acid of the invention, or a fragment thereof may comprise: (a) providing a pair of single stranded oligonucleotides, each of which is at least eight nucleotides in length, complementary to sequences of a nucleic acid of the invention, and wherein the sequences to which the oligonucleotides are complementary are at least ten nucleotides apart; and (b) contacting the oligonucleotides with a sample comprising a nucleic acid comprising the nucleic acid of the invention under conditions which permit amplification of the region located between the pair of oligonucleotides, thereby amplifying the nucleic acid.

Isd proteins may be expressed from recombinant vectors, host cells containing the recombinant vectors and methods of producing the encoded S. aureus polypeptides. Appropriate vectors may be introduced into host cells using well-known techniques such as infection, transduction, transfection, transvection, electroporation and transformation. The vector may be, for example, a phage, plasmid, viral or retroviral vector. Retroviral vectors may be replication competent or replication defective. In the latter case, viral propagation generally will occur only in complementing host cells.

The vector may contain a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

Preferred vectors comprise cis-acting control regions to the polynucleotide of interest. Appropriate trans-acting factors may be supplied by the host, supplied by a complementing vector or supplied by the vector itself upon introduction into the host.

In certain embodiments, the vectors provide for specific expression, which may be inducible and/or cell type-specific. Particularly preferred among such vectors are those inducible by environmental factors that are easy to manipulate, such as temperature and nutrient additives.

Expression vectors useful in the present invention include chromosomal-, episomal- and virus-derived vectors, e.g., vectors derived from bacterial plasmids, bacteriophage, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan The expression constructs may further contain sites for transcription initiation, termination and, in the transcribed region, a ribosome-binding site for translation. The coding portion of the mature transcripts expressed by the constructs may preferably include a translation-initiating site at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline, kanamycin, or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Sf9 cells; animal cells such as CHO, COS and Bowes melanoma cells; and plant cells. Appropriate culture mediums and conditions for the above-described host cells are known in the art.

Among vectors preferred for use in bacteria include pQE70, pQE60 and pQE9, pQE10 available from Qiagen; pBS vectors, Phagescript vectors, Bluescript vectors, pNH8A, pNH16a, pNH18A, pNH46A available from Stratagene; pET series of vectors available from Novagen; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Among known bacterial promoters suitable for use in the present invention include the E. coli lacI and lacZ promoters, the T3, T5 and T7 promoters, the gpt promoter, the lambda PR and PL promoters, the trp promoter and the xyI/tet chimeric promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals (for example, Davis, et al., Basic Methods in Molecular Biology (1986)).

Transcription of DNA encoding the polypeptides of the present invention by higher eukaryotes may be increased by inserting an enhancer sequence into the vector. Enhancers are cis-acting elements of DNA, usually about from 10 to 300 nucleotides that act to increase transcriptional activity of a promoter in a given host cell-type. Examples of enhancers include the SV40 enhancer, which is located on the late side of the replication origin at nucleotides 100 to 270, the cytomegalovirus early promoter enhancer, the polyoma enhancer on the late side of the replication origin, and adenovirus enhancers.

For secretion of the translated polypeptide into the lumen of the endoplasmic reticulum, into the periplasmic space or into the extracellular environment, appropriate secretion signals may be incorporated into the expressed polypeptide, for example, the amino acid sequence KDEL. The signals may be endogenous to the polypeptide or they may be heterologous signals.

Coding sequences for a polypeptide of interest may be incorporated as a part of a fusion gene including a nucleotide sequence encoding a different polypeptide. The present invention contemplates an isolated nucleic acid comprising a nucleic acid of the invention and at least one heterologous sequence encoding a heterologous peptide linked in frame to the nucleotide sequence of the nucleic acid of the invention so as to encode a fusion protein comprising the heterologous polypeptide. The heterologous polypeptide may be fused to (a) the C-terminus of the polypeptide encoded by the nucleic acid of the invention, (b) the N-terminus of the polypeptide, or (c) the C-terminus and the N-terminus of the polypeptide. In certain instances, the heterologous sequence encodes a polypeptide permitting the detection, isolation, solubilization and/or stabilization of the polypeptide to which it is fused. In still other embodiments, the heterologous sequence encodes a polypeptide selected from the group consisting of a poly-His tag, myc, HA, GST, protein A, protein G, calmodulin-binding peptide, thioredoxin, maltose-binding protein, poly arginine, poly-His-Asp, FLAG, a portion of an immunoglobulin protein, and a transcytosis peptide.

Fusion expression systems can be useful when it is desirable to produce an immunogenic fragment of a polypeptide of the invention. For example, the VP6 capsid protein of rotavirus may be used as an immunologic carrier protein for portions of polypeptide, either in the monomeric form or in the form of a viral particle. The nucleic acid sequences corresponding to the portion of a polypeptide of the invention to which antibodies are to be raised may be incorporated into a fusion gene construct which includes coding sequences for a late vaccinia virus structural protein to produce a set of recombinant viruses expressing fusion proteins comprising a portion of the protein as part of the virion. The Hepatitis B surface antigen may also be utilized in this role as well. Similarly, chimeric constructs coding for fusion proteins containing a portion of a polypeptide of the invention and the poliovirus capsid protein may be created to enhance immunogenicity (see, for example, EP Publication NO: 0259149; and Evans et al., (1989) Nature 339:385; Huang et al., (1988) J. Virol. 62:3855; and Schlienger et al., (1992) J. Virol. 66:2).

Fusion proteins may facilitate the expression and/or purification of proteins. For example, a polypeptide of the invention may be generated as a glutathione-S-transferase (GST) fusion protein. Such GST fusion proteins may be used to simplify purification of a polypeptide of the invention, such as through the use of glutathione-derivatized matrices (see, for example, Current Protocols in Molecular Biology, eds. Ausubel et al., (N.Y.: John Wiley & Sons, 1991)). In another embodiment, a fusion gene coding for a purification leader sequence, such as a poly-(His)/enterokinase cleavage site sequence at the N-terminus of the desired portion of the recombinant protein, may allow purification of the expressed fusion protein by affinity chromatography using a Ni²⁺ metal resin. The purification leader sequence may then be subsequently removed by treatment with enterokinase to provide the purified protein (e.g., see Hochuli et al., (1987) J. Chromatography 411: 177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene may be synthesized by conventional techniques including automated DNA synthesizers. Alternatively, PCR amplification of gene fragments may be carried out using anchor primers which give rise to complementary overhangs between two consecutive gene fragments which may subsequently be annealed to generate a chimeric gene sequence (see, for example, Current Protocols in Molecular Biology, eds. Ausubel et al., John Wiley & Sons: 1992).

In other embodiments, nucleic acids of the invention may be immobilized onto a solid surface, including, plates, microtiter plates, slides, beads, particles, spheres, films, strands, precipitates, gels, sheets, tubing, containers, capillaries, pads, slices, etc. The nucleic acids of the invention may be immobilized onto a chip as part of an array. The array may comprise one or more polynucleotides of the invention as described herein. In one embodiment, the chip comprises one or more polynucleotides of the invention as part of an array of polynucleotide sequences.

Another aspect relates to the use of nucleic acids of the invention in “antisense therapy”. As used herein, antisense therapy refers to administration or in situ generation of oligonucleotide probes or their derivatives which specifically hybridize or otherwise bind under cellular conditions with the cellular mRNA and/or genomic DNA encoding one of the polypeptides of the invention so as to inhibit expression of that polypeptide, e.g., by inhibiting transcription and/or translation. The binding may be by conventional base pair complementary, or, for example, in the case of binding to DNA duplexes, through specific interactions in the major groove of the double helix. In general, antisense therapy refers to the range of techniques generally employed in the art, and includes any therapy which relies on specific binding to ohgonucleotide sequences.

The oligonucleotide may be conjugated to another molecule, e.g., a peptide, hybridization triggered cross-linking agent transport agent, hybridization-triggered cleavage agent, etc. An antisense molecule can be a “peptide nucleic acid” (PNA). PNA refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

An antisense construct of the present invention may be delivered, for example, as an expression plasmid which, when transcribed in the cell, produces RNA which is complementary to at least a unique portion of the mRNA which encodes a polypeptide of the invention. Alternatively, the antisense construct may be an oligonucleotide probe which is generated ex vivo and which, when introduced into the cell causes inhibition of expression by hybridizing with the mRNA and/or genomic sequences encoding a polypeptide of the invention Such oligonucleotide probes may be modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by van der Krol et al., (1988) Biotechniques 6:958-976; and Stein et al., (1988) Cancer Res 48:2659-2668.

In a further aspect, double stranded small interfering RNAs (siRNAs), and methods for administering the same are provided. siRNAs decrease or block gene expression. While not wishing to be bound by theory, it is generally thought that siRNAs inhibit gene expression by mediating sequence specific mRNA degradation. RNA interference (RNAi) is the process of sequence-specific, post-transcriptional gene silencing, particularly in animals and plants, initiated by double-stranded RNA (dsRNA) that is homologous in sequence to the silenced gene (Elbashir et al. Nature 2001; 411(6836): 494-8). Accordingly, it is understood that siRNAs and long dsRNAs having substantial sequence identity to all or a portion of a polynucleotide of the present invention may be used to inhibit the expression of a nucleic acid of the invention.

Alternatively, siRNAs that decrease or block the expression the Isd polypeptides described herein may be determined by testing a plurality of siRNA constructs against the target gene. Such siRNAs against a target gene may be chemically synthesized. The nucleotide sequences of the individual RNA strands are selected such that the strand has a region of complementary to the target gene to be inhibited (i.e., the complementary RNA strand comprises a nucleotide sequence that is complementary to a region of an mRNA transcript that is formed during expression of the target gene, or its processing products, or a region of a (+) strand virus). The step of synthesizing the RNA strand may involve solid-phase synthesis, wherein individual nucleotides are joined end to end through the formation of internucleotide 3′-5′ phosphodiester bonds in consecutive synthesis cycles.

Provided herein are siRNA molecules comprising a nucleotide sequence consisting essentially of a sequence of an isd nucleic acid as described herein. An siRNA molecule may comprise two strands, each strand comprising a nucleotide sequence that is at least essentially complementary to each other, one of which corresponds essentially to a sequence of a target gene. The sequence that corresponds essentially to a sequence of a target gene is referred to as the “sense target sequence” and the sequence that is essentially complementary thereto is referred to as the “antisense target sequence” of the siRNA. The sense and antisense target sequences may be from about 15 to about 30 consecutive nucleotides long; from about 19 to about 25 consecutive nucleotides; from about 19 to 23 consecutive nucleotides or about 19, 20, 21, 22 or 23 nucleotides long. The length of the sense and antisense sequences is determined so that an siRNA having sense and antisense target sequences of that length is capable of inhibiting expression of a target gene, preferably without significantly inducing a host interferon response.

SiRNA target sequences may be predicted using any of the aligorithms provided on the world wide web at the mmcmanus with the extension web.mit.edu/mmcmanus/www/home1.2files/siRNAs.

The sense target sequence may be essentially or substantially identical to the coding or a non-coding portion, or combination thereof, of a target nucleic acid. For example, the sense target sequence may be essentially complementary to the 5′ or 3′ untranslated region, promoter, intron or exon of a target nucleic acid or complement thereof. It can also be essentially complementary to a region encompassing the border between two such gene regions.

The nucleotide base composition of the sense target sequence can be about 50% adenines (As) and thymidines (Ts) and 50% cytidines (Cs) and guanosines (Gs). Alternatively, the base composition can be at least 50% Cs/Gs, e.g., about 60%, 70% or 80% of Cs/Gs. Accordingly, the choice of sense target sequence may be based on nucleotide base composition. Regarding the accessibility of target nucleic acids by siRNAs, such can be determined, e.g., as described in Lee et al. (2002) Nature Biotech. 19:500. This approach involves the use of ohgonucleotides that are complementary to the target nucleic acids as probes to determine substrate accessibility, e.g., in cell extracts. After forming a duplex with the oligonucleotide probe, the substrate becomes susceptible to RNase H. Therefore, the degree of RNase H sensitivity to a given probe as determined, e.g., by PCR, reflects the accessibility of the chosen site, and may be of predictive value for how well a corresponding siRNA would perform in inhibiting transcription from this target gene. One may also use algorithms identifying primers for polymerase chain reaction (PCR) assays or for identifying antisense oligonucleotides for identifying first target sequences.

The sense and antisense target sequences are preferably sufficiently complementary, such that an siRNA comprising both sequences is able to inhibit expression of the target gene, i.e., to mediate RNA interference. For example, the sequences may be sufficiently complementary to permit hybridization under the desired conditions, e.g., in a cell. Accordingly, the sense and antisense target sequences may be at least about 95%, 97%, 98%, 99% or 100% identical and may, e.g., differ in at most 5, 4, 3, 2, 1 or 0 nucleotides.

Sense and antisense target sequences are also preferably sequences that are not likely to significantly interact with sequences other than the target nucleic acid or complement thereof. This can be confirmed by, e.g., comparing the chosen sequence to the other sequences in the genome of the target cell. Sequence comparisons can be performed according to methods known in the art, e.g., using the BLAST algorithm, further described herein. Of course, small scale experiments can also be performed to confirm that a particular first target sequence is capable of specifically inhibiting expression of a target nucleic acid and essentially not that of other genes.

siRNAs may also comprise sequences in addition to the sense and antisense sequences. For example, an siRNA may be an RNA duplex consisting of two strands of RNA, in which at least one strand has a 3′ overhang. The other strand can be blunt-ended or have an overhang. In the embodiment in which the RNA molecule is double stranded and both strands comprise an overhang, the length of the overhangs may be the same or different for each strand. In a particular embodiment, an siRNA comprises sense and antisense sequences, each of which are on one RNA strand, consisting of about 19-25 nucleotides which are paired and which have overhangs of from about 1 to about 3, particularly about 2, nucleotides on both 3′ ends of the RNA. In order to further enhance the stability of the RNA of the present invention, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly may also enhance the nuclease resistance of the overhang at least in tissue culture medium. RNA strands of siRNAs may have a 5′ phosphate and a 3′ hydroxyl group.

In one embodiment, an siRNA molecule comprises two strands of RNA forming a duplex. In another embodiment, an siRNA molecule consists of one RNA strand forming a hairpin loop, wherein the sense and antisense target sequences hybridize and the sequence between the two target sequences is a spacer sequence that essentially forms the loop of the hairpin structure. The spacer sequence may be any combination of nucleotides and any length provided that two complementary oligonucleotides linked by a spacer having this sequence can form a hairpin structure, wherein at least part of the spacer forms the loop at the closed end of the hairpin. For example, the spacer sequence can be from about 3 to about 30 nucleotides; from about 3 to about 20 nucleotides; from about 5 to about 15 nucleotides; from about 5 to about 10 nucleotides; or from about 3 to about 9 nucleotides. The sequence can be any sequence, provided that it does not interfere with the formation of a hairpin structure. In particular, the spacer sequence is preferably not a sequence having any significant homology to the first or the second target sequence, since this might interfere with the formation of a hairpin structure. The spacer sequence is also preferably not similar to other sequences, e.g., genomic sequences of the cell into which the nucleic acid will be introduced, since this may result in undesirable effects in the cell.

A person of skill in the art will understand that when referring to a nucleic acid, e.g., an RNA, the RNA may comprise or consist of naturally occurring nucleotides or of nucleotide derivatives that provide, e.g., more stability to the nucleic acid. Any derivative is permitted provided that the nucleic acid is capable of functioning in the desired fashion For example, an siRNA may comprise nucleotide derivatives provided that the siRNA is still capable of inhibiting expression of the target gene.

For example, siRNAs may include one or more modified base and/or a backbone modified for stability or for other reasons. For example, the phosphodiester linkages of natural RNA may be modified to include at least one of a nitrogen or sulphur heteroatom. Moreover, siRNA comprising unusual bases, such as inosine, or modified bases, such as tritylated bases, to name just two examples, can be used in the invention It will be appreciated that a great variety of modifications have been made to RNA that serve many useful purposes known to those of skill in the art. The term siRNA as it is employed herein embraces such chemically, enzymatically or metabolically modified forms of siRNA, provided that it is derived from an endogenous template.

There is no limitation on the manner in which an siRNA may be synthesised. Thus, it may synthesized in vitro or in vivo, using manual and/or automated procedures. In vitro synthesis may be chemical or enzymatic, for example using cloned RNA polymerase (e.g., T3, T7, SP6) for transcription of a DNA (or cDNA) template, or a mixture of both. SiRNAs may also be prepared by synthesizing each of the two strands, e.g., chemically, and hybridizing the two strands to form a duplex. In vivo, the siRNA may be synthesized using recombinant techniques well known in the art (see e.g., Sambrook et al., Molecular Cloning: A Laboratory Manual, Second Edition (1989); DNA Cloning, Volumes I and II (D. N Glover ed. 1985); Oligonucleotide Synthesis (M. J. Gait ed, 1984); Nucleic Acid Hybridisation (B. D. Hames & S. J. Higgins eds. 1984); Transcription and Translation (B. D. Hames & S. J. Higgins eds. 1984); Animal Cell Culture (R. I. Freshney ed. 1986); Immobilised Cells and Enzymes (IRL Press, 1986); B. Perbal, A Practical Guide to Molecular Cloning (1984); the series, Methods in Enzymology (Academic Press, Inc.); Gene Transfer Vectors for Mammalian Cells (J. H. Miller and M. P. Calos eds. 1987, Cold Spring Harbor Laboratory), Methods in Enzymology Vol. 154 and Vol. 155 (Wu and Grossman, and Wu, eds., respectively), Mayer and Walker, eds. (1987), Immunochemical Methods in Cell and Molecular Biology (Academic Press, London), Scopes, (1987), Protein Purification: Principles and Practice, Second Edition (Springer-Verlag, N.Y.), and Handbook of Experimental Immunology, Volumes I-IV (D. M. Weir and C. C. Blackwell eds 1986). For example, bacterial cells can be transformed with an expression vector which comprises the DNA template from which the siRNA is to be derived.

If synthesized outside the cell, the siRNA may be purified prior to introduction into the cell. Purification may be by extraction with a solvent (such as phenol/chloroform) or resin, precipitation (for example in ethanol), electrophoresis, chromatography, or a combination thereof. However, purification may result in loss of siRNA and may therefore be minimal or not carried out at all. The siRNA may be dried for storage or dissolved in an aqueous solution, which may contain buffers or salts to promote annealing, and/or stabilization of the RNA strands.

The double-stranded structure may be formed by a single self-complementary RNA strand or two separate complementary RNA strands.

It is known that mammalian cells can respond to extracellular siRNA and therefore may have a transport mechanism for dsRNA (Asher et al. (1969) Nature 223 715-717). Thus, siRNA may be administered extracellularly into a cavity, interstitial space, into the circulation of a mammal, or introduced orally. Methods for oral introduction include direct mixing of the RNA with food of the mammal, as well as engineered approaches in which a species that is used as food is engineered to express the RNA, then fed to the mammal to be affected. For example, food bacteria, such as Lactococcus lactis, may be transformed to produce the dsRNA (see WO93/17117, WO97/14806). Vascular or extravascular circulation, the blood or lymph systems and the cerebrospinal fluid are sites where the RNA may be injected.

RNA may be introduced into the cell intracellularly. Physical methods of introducing nucleic acids may also be used in this respect. siRNA may be administered using the microinjection techniques described in Zemicka-Goetz et al. (1997) Development 124, 1133-1137 and Wianny et al. (1998) Chromosoma 107, 430-439.

Other physical methods of introducing nucleic acids intracellularly include bombardment by particles covered by the siRNA, for example gene gun technology in which the siRNA is immobilized on gold particles and fired directly at the site of wounding. Thus, the invention provides the use of an siRNA in a gene gun for inhibiting the expression of a target gene. Further, there is provided a composition suitable for gene gun therapy comprising an siRNA and gold particles. An alternative physical method includes electroporation of cell membranes in the presence of the siRNA. This method permits RNAi on a large scale. Other methods known in the art for introducing nucleic acids to cells may be used, such as lipid-mediated carrier transport, chemical-mediated transport, such as calcium phosphate, and the like. siRNA may be introduced along with components that perform one or more of the following activities: enhance RNA uptake by the cell, promote annealing of the duplex strands, stabilize the annealed strands, or otherwise increase inhibition of the target gene.

Any known gene therapy technique can be used to administer the RNA. A viral construct packaged into a viral particle would accomplish both efficient introduction of an expression construct into the cell and transcription of siRNA encoded by the expression construct. Thus, siRNA can also be produced inside a cell. Vectors, e.g., expression vectors that comprise a nucleic acid encoding one or the two strands of an siRNA molecule may be used for that purpose. The nucleic acid may further comprise an antisense sequence that is essentially complementary to the sense target sequence. The nucleic acid may further comprise a spacer sequence between the sense and the antisense target sequence. The nucleic acid may further comprise a promoter for directing expression of the sense and antisense sequences in a cell, e.g., an RNA Polymerase II or III promoter and a transcriptional termination signal. The sequences may be operably linked.

In one embodiment a nucleic acid comprises an RNA coding region (e.g., sense or antisense target sequence) operably linked to an RNA polymerase III promoter. The RNA coding region can be immediately followed by a pol III terminator sequence, which directs termination of RNA synthesis by pol III. The pol III terminator sequences generally have 4 or more consecutive thymidine (“T”) residues. In a preferred embodiment, a cluster of 5 consecutive T residues is used as the terminator by which pol III transcription is stopped at the second or third T of the DNA template, and thus only 2 to 3 uridine (“U”) residues are added to the 3′ end of the coding sequence. A variety of pol III promoters can be used with the invention, including for example, the promoter fragments derived from H1 RNA genes or U6 snRNA genes of human or mouse origin or from any other species. In addition, pol III promoters can be modified/engineered to incorporate other desirable properties such as the ability to be induced by small chemical molecules, either ubiquitously or in a tissue-specific manner. For example, in one embodiment the promoter may be activated by tetracycline. In another embodiment the promoter may be activated by IPTG (lacI system).

siRNAs can be produced in cells by transforming cells with two nucleic acids, e.g., vectors, each nucleic acid comprising an expressing cassette, each expression cassette comprising a promoter, an RNA coding sequence (one being a sense target sequence and the other being an antisense target sequence) and a termination signal. Alternatively, a single nucleic acid may comprise these two expression cassettes. In yet another embodiment, a nucleic acid encodes a single stranded RNA comprising a sense target sequence linked to a spacer linked to an antisense target sequence. The nucleic acids may be present in a vector, such as an expression vector, e.g., a eukaryotic expression vector that allows expression of the sense and antisense target sequences in cells into which it is introduced.

Vectors for producing siRNAs are described, e.g., in Paul et al. (2002) Nature Biotechnology 29:505; Xia et al. (2002) Nature Biotechnology 20:1006; Zeng et al. (2002) Mol. Cell 9:1327; Thijn et al. (2002) Science 296:550; BMC Biotechnol. August 2002 28;2(1):15; Lee et al. (2002) Nature Biotechnology 19: 500; McManus et al. (2002) RNA 8:842; Miyagishi et al. (2002) Nature Biotechnology 19:497; Sui et al. (2002) PNAS 99:5515; Yu et al. (2002) PNAS 99:6047; Shi et al. (2003) Trends Genet. 19(1):9; Gaudilliere et al. (2002) J. Biol. Chem. 277(48):46442; US2002/0182223; US 2003/0027783; WO 01/36646 and WO 03/006477. Vectors are also available commercially. For example, the pSilencer is available from Gene Therapy Systems, Inc. and pSUPER RNAi system is available from Oligoengine.

Also provided herein are compositions comprising one or more siRNA or nucleic acid encoding an RNA coding region of an siRNA. Compositions may be pharmaceutical compositions and comprise a pharmaceutically acceptable carrier. Compositions may also be provided in a device for administering the composition in a cell or in a subject. For example a composition may be present in a syringe or on a stent. A composition may also comprise agents facilitating the entry of the siRNA or nucleic acid into a cell. In general, the oligonucleotides may be synthesized using protocols known in the art, for example, as described in Caruthers et al., Methods in Enzymology (1992) 211:3-19; Thompson et al., International PCT Publication No. WO 99/54459; Wincott et al., Nucl. Acids Res. (1995) 23:2677-2684; Wincott et al., Methods Mol. Bio., (1997) 74:59; Brennan et al., Biotechnol. Bioeng. (1998) 61:33-45; and Brennan, U.S. Pat. No. 6,001,311; each of which is hereby incorporated by reference in its entirety herein. In general, the synthesis of oligonucleotides involves conventional nucleic acid protecting and coupling groups, such as dimethoxytrityl at the 5′-end, and phosphoramidites at the 3′-end. In a non-limiting example, small scale syntheses are conducted on a Expedite 8909 RNA synthesizer sold by Applied Biosystems, Inc. (Weiterstadt, Germany), using ribonucleoside phosphoramidites sold by ChemGenes Corporation (Ashland Technology Center, 200 Homer Avenue, Ashland, Mass. 01721, USA). Alternatively, syntheses can be performed on a 96-well plate synthesizer, such as the instrument produced by Protogene (Palo Alto, Calif., USA), or by methods such as those described in Usman et al., J. Am. Chem. Soc. (1987) 109:7845; Scaringe et al., Nucl. Acids Res. (1990) 18:5433; Wincott et al., Nucl. Acids Res. (1990) 23:2677-2684; and Wincott et al., Methods Mol. Bio. (1997) 74:59, each of which is hereby incorporated by reference in its entirety.

The nucleic acid molecules of the present invention may be synthesized separately and dsRNAs may be formed post-synthetically, for example, by ligation (Moore et al., Science (1992) 256:9923; Draper et al., International PCT publication No. WO 93/23569; Shabarova et al., Nucl. Acids Res. (1991) 19:4247; Bellon et al., Nucleosides & Nucleotides (1997) 16:951; and Bellon et al., Bioconjugate Chem. (1997) 8:204; or by hybridization following synthesis and/or deprotection. The nucleic acid molecules can be purified by gel electrophoresis using conventional methods or can be purified by high pressure liquid chromatography (HPLC; see Wincott et al., supra, the totality of which is hereby incorporated herein by reference) and re-suspended in water.

In another embodiment, the level of a particular mRNA or polypeptide in a cell is reduced by introduction of a ribozyme into the cell or nucleic acid encoding such. Ribozyme molecules designed to catalytically cleave mRNA transcripts can also be introduced into, or expressed, in cells to inhibit target gene expression (see, e.g., Sarver et al., 1990, Science 247:1222-1225 and U.S. Pat. No. 5,093,246). One commonly used ribozyme motif is the hammerhead, for which the substrate sequence requirements are minimal. Design of the hammerhead ribozyme is disclosed in Usman et al., Current Opin. Struct. Biol. (1996) 6:527-533. Usman also discusses the therapeutic uses of ribozymes. Ribozymes can also be prepared and used as described in Long et al., FASEB J. (1993) 7:25; Symons, Ann. Rev. Biochem. (1992) 61:641; Perrotta et al., Biochem. (1992) 31:16-17; Ojwang et al., Proc. Natl. Acad. Sci. (USA) (1992) 89:10802-10806; and U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337 and Koizumi et al., Nucleic Acid Res. (1989) 17:7059-7071. Preparation and use of ribozyme fragments in a hammerhead structure are also described by Koizumi et al., Nucleic Acids Res. (1989) 17:7059-7071. Preparation and use of ribozyme fragments in a hairpin structure are described by Chowrira and Burke, Nucleic Acids Res. (1992) 20:2835. Ribozymes can also be made by rolling transcription as described in Daubendiek and Kool, Nat. Biotechnol. (1997) 15(3):273-277.

Gene expression can be reduced by targeting deoxyribonucleotide sequences complementary to the regulatory region of the target gene (i.e., the gene promoter and/or enhancers) to form triple helical structures that prevent transcription of the gene in target cells in the body. (See generally, Helene (1991) Anticancer Drug Des., 6(6):569-84; Helene et al. (1992) Ann. N.Y. Acad. Sci., 660:27-36; and Maher (1992) Bioassays 14(12):807-15).

In a further embodiment, RNA aptamers can be introduced into or expressed in a cell. RNA aptamers are specific RNA ligands for proteins, such as for Tat and Rev RNA (Good et al. (1997) Gene Therapy 4: 45-54) that can specifically inhibit their translation.

4. Isd Polypeptides

The S. aureus polypeptides, including IsdA (SEQ ID NO: 3), IsdB (SEQ ID NO: 6), and IsdC (SEQ ID NO: 9) (FIGS. 1-3), described herein, include naturally purified products, products of chemical synthetic procedures, and products produced by recombinant techniques from a prokaryotic or eukaryotic host cell, including for example, bacterial, yeast, higher plant, insect, and mammalian cells. In certain, embodiments, the polypeptides disclosed herein inhibit the function of Isd polypeptides.

Polypeptides may also comprise, consist of or consist essentially of any of the amino acid sequences described herein. Yet other polypeptides comprise, consist of or consist essentially of an amino acid sequence that has at least about 70%, 80%, 90%, 95%, 98% or 99% identity or homology with an Isd polypeptide. For example, polypeptides that differ from a sequence in a naturally occurring Isd protein in about 1, 2, 3, 4, 5 or more amino acids are also contemplated. The differences may be substitutions, e.g., conservative substitutions, deletions or additions. The differences are preferably in regions that are not significantly conserved among different species. Such regions may be identified by aligning the amino acid sequences of Isd proteins from various species. These amino acids can be substituted, e.g., with those found in another species. Other amino acids that may be substituted, inserted or deleted at these or other locations can be identified by mutagenesis studies coupled with biological assays.

Proteins may also comprise one or more non-naturally occurring amino acids. For example, nonclassical amino acids or chemical amino acid analogs can be introduced as a substitution or addition into proteins. Non-classical amino acids include, but are not limited to, the D-isomers of the common amino acids, 2,4-diaminobutyric acid, alpha-amino isobutyric acid, 4-aminobutyric acid, Abu, 2-amino butyric acid, gamma-Abu, epsilon-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, beta-alanine, fluoro-amino acids, designer amino acids such as beta-methyl amino acids, Calpha-methyl amino acids, Nalpha-methyl amino acids, and amino acid analogs in general. Furthermore, the amino acid can be D (dextrorotary) or L (levorotary).

Yet other proteins that are encompassed herein are those that comprise modified amino acids. Exemplary proteins are derivative proteins that may be one modified by glycosylation, pegylation, phosphorylation or any similar process that retains at least one biological function of the protein from which it was derived.

Proteins may be used as a substantially pure preparation, e.g., wherein at least about 90% of the protein in the preparation are the desired protein. Compositions comprising at least about 50%, 60%, 70%, or 80% of the desired protein may also be used.

The S. aureus polypeptides can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography and high performance liquid chromatography (“HPLC”) is employed for purification. Proteins may be used as a substantially pure preparation, e.g., wherein at least about 90% of the protein in the preparation are the desired protein. Compositions comprising at least about 50%, 60%, 70%, or 80% of the desired protein may also be used.

Proteins may be denatured or non-denatured and may be aggregated or non-aggregated as a result thereof. Proteins can be denatured according to methods known in the art.

In certain embodiments, an Isd polypeptide described herein may be a fusion protein containing a domain which increases its solubility and/or facilitates its purification, identification, detection, and/or structural characterization. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type III secretion system-targeting peptides, transcytosis domains, nuclear localization signals, etc. In various embodiments, a polypeptide of the invention may comprise one or more heterologous fusions. Polypeptides may contain multiple copies of the same fusion domain or may contain fusions to two or more different domains. The fusions may occur at the N-terminus of the polypeptide, at the C-terminus of the polypeptide, or at both the N— and C-terminus of the polypeptide. It is also within the scope of the invention to include linker sequences between a polypeptide of the invention and the fusion domain in order to facilitate construction of the fusion protein or to optimize protein expression or structural constraints of the fusion protein. In another embodiment, the polypeptide may be constructed so as to contain protease cleavage sites between the fusion polypeptide and polypeptide of the invention in order to remove the tag after protein expression or thereafter. Examples of suitable endoproteases, include, for example, Factor Xa and TEV proteases. A protein may also be fused to a signal sequence. For example, when prepared recombinantly, a nucleic acid encoding the peptide may be linked at its 5′ end to a signal sequence, such that the protein is secreted from the cell.

In certain embodiments, polypeptides of the invention may be synthesized chemically, ribosomally in a cell free system, or ribosomally within a cell. Chemical synthesis of polypeptides of the invention may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full length ligation product having a native peptide bond at the ligation site. Full length ligation products are chemically identical to proteins produced by cell free synthesis. Full length ligation products may be refolded and/or oxidized, as allowed, to form native disulfide-containing protein molecules. (see e.g., U.S. Pat. Nos. 6,184,344 and 6,174,530; and Muir et al., Curr. Opin. Biotech. (1993): vol. 4, p 420; Miller et al., Science (1989): vol. 246, p 1149; Wlodawer et al., Science (1989): vol. 245, p 616; Huang et al., Biochemistry (1991): vol. 30, p 7402; Schnolzer, et al., Int. J. Pept. Prot. Res. (1992): vol. 40, p 180-193; Rajarathnam et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; Wallace et al., J. Biol. Chem. (1992): vol. 267, p 3852; Abrahmsen et al., Biochemistry (1991): vol. 30, p 4151; Chang, et al., Proc. Natl. Acad. Sci. USA (1994) 91: 12544-12548; Schnlzer et al., Science (1992): vol., 3256, p 221; and Akaji et al., Chem. Pharm. Bull. (Tokyo) (1985) 33: 184).

In certain embodiments, it may be advantageous to provide naturally-occurring or experimentally-derived homologs of a polypeptide of the invention. Such homologs may function in a limited capacity as a modulator to promote or inhibit a subset of the biological activities of the naturally-occurring form of the polypeptide. Thus, specific biological effects may be elicited by treatment with a homolog of limited function, and with fewer side effects relative to treatment with agonists or antagonists which are directed to all of the biological activities of a polypeptide of the invention. For instance, antagonistic homologs may be generated which interfere with the ability of the wild-type polypeptide of the invention to associate with certain proteins, but which do not substantially interfere with the formation of complexes between the native polypeptide and other cellular proteins.

Polypeptides may be derived from the full-length polypeptides of the invention. Isolated peptidyl portions of those polypeptides may be obtained by screening polypeptides recombinantly produced from the corresponding fragment of the nucleic acid encoding such polypeptides. In addition, fragments may be chemically synthesized using techniques known in the art such as conventional Merrifield solid phase f-Moc or t-Boc chemistry. For example, proteins may be arbitrarily divided into fragments of desired length with no overlap of the fragments, or may be divided into overlapping fragments of a desired length. The fragments may be produced (recombinantly or by chemical synthesis) and tested to identify those peptidyl fragments having a desired property, for example, the capability of functioning as a modulator of the polypeptides of the invention. In an illustrative embodiment, peptidyl portions of a protein of the invention may be tested for binding activity, as well as inhibitory ability, by expression as, for example, thioredoxin fusion proteins, each of which contains a discrete fragment of a protein of the invention (see, for example, U.S. Pat. Nos. 5,270,181 and 5,292,646; and PCT publication WO94/02502).

In another embodiment, truncated polypeptides may be prepared. Truncated polypeptides have from 1 to 20 or more amino acid residues removed from either or both the N— and C-termini. Such truncated polypeptides may prove more amenable to expression, purification or characterization than the full-length polypeptide. For example, truncated polypeptides may prove more amenable than the full-length polypeptide to crystallization, to yielding high quality diffracting crystals or to yielding an HSQC spectrum with high intensity peaks and minimally overlapping peaks. In addition, the use of truncated polypeptides may also identify stable and active domains of the full-length polypeptide that may be more amenable to characterization.

It is also possible to modify the structure of the polypeptides of the invention for such purposes as enhancing therapeutic or prophylactic efficacy, or stability (e.g., ex vivo shelf life, resistance to proteolytic degradation in vivo, etc.). Such modified polypeptides, when designed to retain at least one activity of the naturally-occurring form of the protein, are considered “functional equivalents” of the polypeptides described in more detail herein Such modified polypeptides may be produced, for instance, by amino acid substitution deletion, or addition, which substitutions may consist in whole or part by conservative amino acid substitutions.

For instance, it is reasonable to expect that an isolated conservative amino acid substitution, such as replacement of a leucine with an isoleucine or valine, an aspartate with a glutamate, a threonine with a serine, will not have a major affect on the biological activity of the resulting molecule. Whether a change in the amino acid sequence of a polypeptide results in a functional homolog may be readily determined by assessing the ability of the variant polypeptide to produce a response similar to that of the wild-type protein. Polypeptides in which more than one replacement has taken place may readily be tested in the same manner.

Methods of generating sets of combinatorial mutants of polypeptides of the invention are provided, as well as truncation mutants, and is especially useful for identifying potential variant sequences (e.g., homologs). The purpose of screening such combinatorial libraries is to generate, for example, homologs which may modulate the activity of a polypeptide of the invention, or alternatively, which possess novel activities altogether. Combinatorially-derived homologs may be generated which have a selective potency relative to a naturally-occurring protein. Such homologs may be used in the development of therapeutics.

Likewise, mutagenesis may give rise to homologs which have intracellular half-lives dramatically different than the corresponding wild-type protein. For example, the altered protein may be rendered either more stable or less stable to proteolytic degradation or other cellular process which result in destruction of, or otherwise inactivation of the protein. Such homologs, and the genes which encode them, may be utilized to alter protein expression by modulating the half-life of the protein. As above, such proteins may be used for the development of therapeutics or treatment.

In similar fashion, protein homologs may be generated by the present combinatorial approach to act as antagonists, in that they are able to interfere with the activity of the corresponding wild-type protein.

In a representative embodiment of this method, the amino acid sequences for a population of protein homologs are aligned, preferably to promote the highest homology possible. Such a population of variants may include, for example, homologs from one or more species, or homologs from the same species but which differ due to mutation. Amino acids which appear at each position of the aligned sequences are selected to create a degenerate set of combinatorial sequences. In certain embodiments, the combinatorial library is produced by way of a degenerate library of genes encoding a library of polypeptides which each include at least a portion of potential protein sequences. For instance, a mixture of synthetic oligonucleotides may be enzymatically ligated into gene sequences such that the degenerate set of potential nucleotide sequences are expressible as individual polypeptides, or alternatively, as a set of larger fusion proteins (e.g. for phage display).

There are many ways by which the library of potential homologs may be generated from a degenerate oligonucleotide sequence. Chemical synthesis of a degenerate gene sequence may be carried out in an automatic DNA synthesizer, and the synthetic genes may then be ligated into an appropriate vector for expression. One purpose of a degenerate set of genes is to provide, in one mixture, all of the sequences encoding the desired set of potential protein sequences. The synthesis of degenerate oligonucleotides is well known in the art (see for example, Narang (1983) Tetrahedron 39:3; Itakura et al. (1981) Recombinant DNA, Proc. 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al., (1983) Nucleic Acid Res. 11:477). Such techniques have been employed in the directed evolution of other proteins (see, for example, Scott et al. (1990) Science 249:386-390; Roberts et al. (1992) PNAS USA 89:2429-2433; Devlin et al. (1990) Science 249: 404-406; Cwirla et al. (1990) PNAS USA 87: 6378-6382; as well as U.S. Pat. Nos. 5,223,409, 5,198,346, and 5,096,815).

Alternatively, other forms of mutagenesis may be utilized to generate a combinatorial library. For example, protein homologs (both agonist and antagonist forms) may be generated and isolated from a library by screening using, for example, alanine scanning mutagenesis and the like (Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al., (1989) Science 244:1081-1085), by linker scanning mutagenesis (Gustin et al. (1993) Virology 193:653-660; Brown et al. (1992) Mol. Cell Biol. 12:2644-2652; McKnight et al. (1982) Science 232:316); by saturation mutagenesis (Meyers et al. (1986) Science 232:613); by PCR mutagenesis (Leung et al. (1989) Method Cell Mol Biol 1:11-19); or by random mutagenesis (Miller et al. (1992). A Short Course in Bacterial Genetics, CSHL Press, Cold Spring Harbor, N.Y. and Greener et al. (1994) Strategies in Mol Biol 7:32-34). Linker scanning mutagenesis, particularly in a combinatorial setting, is an attractive method for identifying truncated forms of proteins that are bioactive.

A wide range of techniques are known in the art for screening gene products of combinatorial libraries made by point mutations and truncations, and for screening cDNA libraries for gene products having a certain property. Such techniques will be generally adaptable for rapid screening of the gene libraries generated by the combinatorial mutagenesis of protein homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

In an illustrative embodiment of a screening assay, candidate combinatorial gene products are displayed on the surface of a cell and the ability of particular cells or viral particles to bind to the combinatorial gene product is detected in a “panning assay”. For instance, the gene library may be cloned into the gene for a surface membrane protein of a bacterial cell (Ladner et al., WO 88/06630; Fuchs et al., (1991) Bio/Technology 9:1370-1371; and Goward et al., (1992) TIBS 18:136-140), and the resulting fusion protein detected by panning, e.g. using a fluorescently labeled molecule which binds the cell surface protein, e.g., FITC-substrate, to score for potentially functional homologs. Cells may be visually inspected and separated under a fluorescence microscope, or, when the morphology of the cell permits, separated by a fluorescence-activated cell sorter. This method may be used to identify substrates or other polypeptides that can interact with a polypeptide of the invention.

In similar fashion, the gene library may be expressed as a fusion protein on the surface of a viral particle. For instance, in the filamentous phage system, foreign peptide sequences may be expressed on the surface of infectious phage, thereby conferring two benefits. First, because these phage may be applied to affinity matrices at very high concentrations, a large number of phage may be screened at one time. Second, because each infectious phage displays the combinatorial gene product on its surface, if a particular phage is recovered from an affinity matrix in low yield, the phage may be amplified by another round of infection. The group of almost identical E. coli filamentous phages M13, fd, and fl are most often used in phage display libraries, as either of the phage gII or gVIII coat proteins may be used to generate fusion proteins without disrupting the ultimate packaging of the viral particle (Ladner et al., PCT publication WO 90/02909; Garrard et al., PCT publication WO 92/09690; Marks et al., (1992) J. Biol. Chem. 267:16007-16010; Griffiths et al., (1993) EMBO J. 12:725-734; Clackson et al., (1991) Nature 352:624-628; and Barbas et al., (1992) PNAS USA 89:4457-4461). Other phage coat proteins may be used as appropriate.

The polypeptides disclosed herein may be reduced to generate mimetics, e.g. peptide or non-peptide agents, which are able to mimic binding of the authentic protein to another cellular partner. Such mutagenic techniques as described above, as well as the thioredoxin system, are also particularly useful for mapping the determinants of a protein which participates in a protein-protein interaction with another protein. To illustrate, the critical residues of a protein which are involved in molecular recognition of a substrate protein may be determined and used to generate peptidomimetics that may bind to the substrate protein. The peptidomimetic may then be used as an inhibitor of the wild-type protein by binding to the substrate and covering up the critical residues needed for interaction with the wild-type protein, thereby preventing interaction of the protein and the substrate. By employing, for example, scanning mutagenesis to map the amino acid residues of a protein which are involved in binding a substrate polypeptide, peptidomimetic compounds may be generated which mimic those residues in binding to the substrate.

For instance, derivatives of the Isd proteins described herein may be chemically modified peptides and peptidomimetics. Peptidomimetics are compounds based on, or derived from, peptides and proteins. Peptidomimetics can be obtained by structural modification of known peptide sequences using unnatural amino acids, conformational restraints, isosteric replacement, and the like. The subject peptidomimetics constitute the continum of structural space between peptides and non-peptide synthetic structures; peptidomimetics may be useful, therefore, in delineating pharmacophores and in helping to translate peptides into nonpeptide compounds with the activity of the parent peptides.

Moreover, mimetopes of the subject peptides can be provided. Such peptidomimetics can have such attributes as being non-hydrolyzable (e.g., increased stability against proteases or other physiological conditions which degrade the corresponding peptide), increased specificity and/or potency for stimulating cell differentiation. For illustrative purposes, non-hydrolyzable peptide analogs of such residues may be generated using benzodiazepine (e.g., see Freidinger et al., in Peptides: Chemistry and Biology, G. R. Marshall ed., ESCOM Publisher: Leiden, Netherlands, 1988), azepine (e.g., see Huffman et al., in Peptides: Chemistry and Biology, G. R. Marshall ed., ESCOM Publisher: Leiden, Netherlands, 1988), substituted gamma lactam rings (Garvey et al., in Peptides: Chemistry and Biology, G. R. Marshall ed., ESCOM Publisher: Leiden, Netherlands, 1988), keto-methylene pseudopeptides (Ewenson et al., (1986) J. Med. Chem. 29:295; and Ewenson et al., in Peptides: Structure and Function (Proceedings of the 9th American Peptide Symposium) Pierce Chemical Co. Rockland, Ill. 1985), β-turn dipeptide cores (Nagai et al., (1985) Tetrahedron Lett 26:647; and Sato et al. (1986) J Chem Soc Perkin Trans 1:1231), and β-aminoalcohols (Gordon et al. (1985) Biochem Biophys Res Commun 126:419; and Dann et al. (1986) Biochem Biophys Res Commun 134:71).

In addition to a variety of sidechain replacements which can be carried out to generate peptidomimetics, the description specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides. 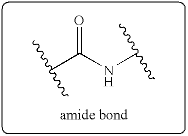
Examples of Surrogates:

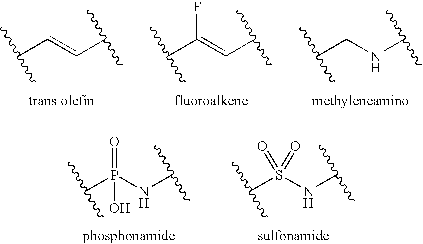

Additionally, peptidomimietics based on more substantial modifications of the backbone of a peptide can be used. Peptidomimetics which fall in this category include (i) retro-inverso analogs, and (ii) N-alkyl glycine analogs (so-called peptoids). 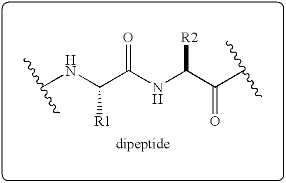
Examples of analogs:

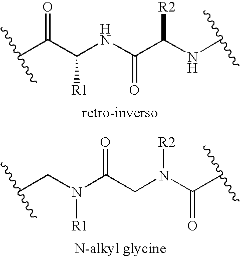

Furthermore, the methods of combinatorial chemistry are being brought to bear, on the development of new peptidomimetics. For example, one embodiment of a so-called “peptide morphing” strategy focuses on the random generation of a library of peptide analogs that comprise a wide range of peptide bond substitutes.

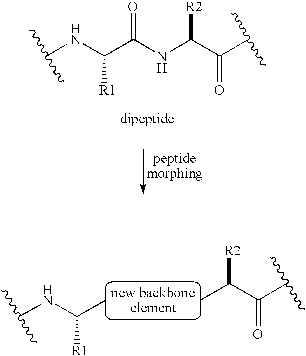

In an exemplary embodiment, the peptidomimetic can be derived as a retro-inverso analog of the peptide. Such retro-inverso analogs can be made according to the methods known in the art, such as that described by the Sisto et al. U.S. Pat. No. 4,522,752. A retro-inverso analog can be generated as described, e.g., in WO 00/01720. It will be understood that a mixed peptide, e.g., including some normal peptide linkages, may be generated. As a general guide, sites which are most susceptible to proteolysis are typically altered, with less susceptible amide linkages being optional for mimetic switching. The final product, or intermediates thereof, can be purified by HPLC.

Peptides may comprise at least one amino acid or every amino acid that is a D stereoisomer. Other peptides may comprise at least one amino acid that is reversed. The amino acid that is reversed may be a D stereoisomer. Every amino acid of a peptide may be reversed and/or every amino acid may be a D stereoisomer.

In another illustrative embodiment, a peptidomimetic can be derived as a retro-enantio analog of a peptide. Retro-enantio analogs such as this can be synthesized with commercially available D-amino acids (or analogs thereof) and standard solid- or solution-phase peptide-synthesis techniques, as described, e.g., in WO 00/01720. The final product may be purified by HPLC to yield the pure retro-enantio analog.

In still another illustrative embodiment, trans-olefin derivatives can be made for the subject peptide. Trans-olefin analogs can be synthesized according to the method of Y. K. Shue et al. (1987) Tetrahedron Letters 28:3225 and as described in WO 00/01720. It is further possible to couple pseudodipeptides synthesized by the above method to other pseudodipeptides, to make peptide analogs with several olefinic functionalities in place of amide functionalities.

Still another class of peptidomimetic derivatives include the phosphonate derivatives. The synthesis of such phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill. 1985).

Many other peptidomimetic structures are known in the art and can be readily adapted for use in the subject peptidomimetics. To illustrate, a peptidomimetic may incorporate the 1-azabicyclo[4.3.0]nonane surrogate (see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem 39:1345-1348). In still other embodiments, certain amino acid residues can be replaced with aryl and bi-aryl moieties, e.g., monocyclic or bicyclic aromatic or heteroaromatic nucleus, or a biaromatic, aromatic-heteroaromatic, or biheteroaromatic nucleus.

The subject peptidomimetics can be optimized by, e.g., combinatorial synthesis techniques combined with high throughput screening.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of inhibiting cell survival and/or tumor growth. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography The three-dimensional structure can then be used to predict structures of potential mimetopes by, for example, computer modelling. The predicted mimetope structures can then be produced by, for example, chemical synthesis, recombinant DNA technology, or by isolating a mimetope from a natural source (e.g., plants, animals, bacteria and fungi).

“Peptides, variants and derivatives thereof” or “peptides and analogs thereof” are included in “peptide therapeutics” and is intended to include any of the peptides or modified forms thereof, e.g., peptidomimetics, described herein. Preferred peptide therapeutics decrease cell survival or increase apoptosis. For example, they may decrease cell survival or increase apoptosis by a factor of at least about 2 fold, 5 fold, 10 fold, 30 fold or 100 fold, as determined, e.g., in an assay described herein.

The activity of an Isd protein, fragment, or variant thereof may be assayed using an appropriate substrate or binding partner or other reagent suitable to test for the suspected activity as described below.

In another embodiment, the activity of a polypeptide may be determined by assaying for the level of expression of RNA and/or protein molecules. Transcription levels may be determined, for example, using Northern blots, hybridization to an oligonucleotide array or by assaying for the level of a resulting protein product. Translation levels may be determined, for example, using Western blotting or by identifying a detectable signal produced by a protein product (e.g., fluorescence, luminescence, enzymatic activity, etc.). Depending on the particular situation, it may be desirable to detect the level of transcription and/or translation of a single gene or of multiple genes.

Alternatively, it may be desirable to measure the overall rate of DNA replication, transcription and/or translation in a cell. In general this may be accomplished by growing the cell in the presence of a detectable metabolite which is incorporated into the resultant DNA, RNA, or protein product. For example, the rate of DNA synthesis may be determined by growing cells in the presence of BrdU which is incorporated into the newly synthesized DNA. The amount of BrdU may then be determined histochemically using an anti-BrdU antibody.

In other embodiments, polypeptides of the invention may be immobilized onto a solid surface, including, microtiter plates, slides, beads, films, etc. The polypeptides of the invention may be immobilized onto a “chip” as part of an array. An array, having a plurality of addresses, may comprise one or more polypeptides of the invention in one or more of those addresses. In one embodiment, the chip comprises one or more polypeptides of the invention as part of an array of polypeptide sequences.

In other embodiments, polypeptides of the invention may be immobilized onto a solid surface, including, plates, microtiter plates, slides, beads, particles, spheres, films, strands, precipitates, gels, sheets, tubing, containers, capillaries, pads, slices, etc. The polypeptides of the invention may be immobilized onto a “chip” as part of an array. An array, having a plurality of addresses, may comprise one or more polypeptides of the invention in one or more of those addresses. In one embodiment, the chip comprises one or more polypeptides of the invention as part of an array.

5. Isd Vaccines

IsdA, IsdB, and IsdC polypeptides are cell surface proteins expressed by S. aureus and are essential for full virulence in vivo (shown using a mouse model of kidney infection). Further, IsdA is immunodominant as anti-IsdA antibodies are detected in convalescent human sera. Thus, the IsdA, IsdB, and/or IsdC polypeptides may be used as a vaccine therapy to treat S. aureus infections.

IsdA, IsdB, and/or IsdC polypeptides or polynucleotides may be formulated into a vaccine and administered to a subject to induce an immune response (e.g. cellular or humoral) against IsdA, IsdB, and/or IsdC in that subject.

An exemplary IsdA protein for inclusion in a vaccine is the full length IsdA polypeptide or an IsdA peptide. In certain embodiments, recombinant IsdA protein will be used in a vaccine. In alternate embodiments, IsdB or IsdC protein used as a vaccine may be full-length IsdB or IsdC, a peptide fragment of IsdB or IsdC, or recombinant IsdB or IsdC protein.

Isd peptides that are antigenic and used as a vaccine may be identified using a variety of methods. In one approach, peptides containing antigenic sequences may be selected on the basis of generally accepted criteria of potential antigenicity and/or exposure. Such criteria include the hydrophilicity and relative antigenic index, as determined by surface exposure analysis of proteins. The determination of appropriate criteria is well-known to one of skill in the art, and has been described, for example, by Hopp et al., Proc Natl Acad Sci USA 1981; 78: 3824-8; Kyte et al., J Mol Biol 1982; 157: 105-32; Emini, J Virol 1985; 55: 836-9; Jameson et al., CA BIOS 1988; 4: 181-6; and Karplus et al., Naturwissenschaften 1985; 72: 212-3. Amino acid domains predicted by these criteria to be surface exposed may be selected preferentially over domains predicted to be more hydrophobic.

Portions of IsdA, IsdB and/or IsdC determined to be antigenic may be chemically synthesized by methods known in the art from individual amino acids. Suitable methods for synthesizing protein fragments are described by Stuart and Young in “Solid Phase Peptide Synthesis,” Second Edition, Pierce Chemical Company (1984).

Alternatively, antigenic linear epitope(s) of IsdA, IsdB or IsdC may be identified by minotope analysis with a corresponding Isd antibody. Briefly, for mimotope analysis a polypeptide will be subdivided into overlapping fragments. For example, overlapping 15 amino acid peptides will be synthesized to cover the entire length of the full-length polypeptide. Each 15 amino acid peptide may overlap by three amino acids. Alternatively, 15 amino acid peptide fragments may be designed in tandem order to cover the entire linear amino acid sequence. Each peptide may then biotinylated and allowed to bind to strepavidin-coated wells in 96-well plates. The reactivity of various antisera may be detected by enzyme-linked immunosorbent assay (ELISA). After blocking non-specific binding, an anti-Isd antibody may be added to each well, followed by the sequential addition of peroxidase-conjugated secondary antibody, and peroxidase substrate. Anti-Isd antibodies may be affinity purified anti-full-length recombinant IsdA or affinity purified anti-IsdA peptide. Alternatively, anti-Isd antibodies may be against IsdB or IsdC. The optical density of each well may be read at 450 nm and duplicate or triplicate wells may be averaged. The average value obtained from a similar ELISA using control serum (i.e., preimmune serum) may be subtracted from the test immunoglobulin values and the resultant values may be plotted to determine which linear epitopes are recognized by the immunoglobulin(s).

Further, competitive binding assays using synthetic peptides representing linear eptitopes may be used to determine antigenic fragments. In certain embodiments, antigenic fragments may inhibit uptake of labeled iron.

Also provided herein are DNA vaccines comprising nucleotide sequences, which encode IsdA, IsdB, and/or IsdC peptides. Exemplary DNA vaccines encode two or more IsdA peptides. Alternate DNA vaccines may encode two or more IsdB or IsdC peptides or any combination of two or more IsdA, IsdB, or IsdC peptides. The efficacy of candidate vaccines (peptide or DNA) may be tested in appropriate animal models such as rats, mice, guinea pigs, monkeys and baboons. A protective or positive effect of the vaccine should be reflected by reduced fertility in the experimental animals.

Nucleic acids encoding IsdA, IsdB, or IsdC immunogens may be obtained by polymerase chain reaction (PCR), amplification of gene segments from genomic DNA, cDNA, RNA (e.g. by RT-PCR), or cloned sequences. PCR primers are chosen, based on the known sequences of the genes or cDNA, so that they result in the amplification of relatively unique fragments. Computer programs may be used in the design of primers with required specificity and optimal amplification purposes. See e.g., Oligo version 5.0 (National Biosciences). Factors which apply to the design and selection of primers for amplification are described for example, by Rylchik, W. (1993) “Selection of Primers for Polymerase Chain Reaction.” In Methods in Molecular Biology, vol. 15, White B. ed., Humana Press, Totowa, N.J. Sequences may be obtained from GenBank or other public sources. Alternatively, the nucleic acids of this invention may also be synthesized by standard methods known in the art, e.g. by use of an automated DNA synthesizer (such synthesizers are commercially available from Biosearch, Applied Biosystems, etc). Suitable cloning vectors for expressing Isd polypeptides in a host or in a cell may be constructed according to standard techniques as described above.

Isd immunogens may alternatively be prepared from enzymatic cleavage of intact Isd polypeptides. Examples of proteolytic enzymes include, but are not limited to, trypsin, chymotrypsin, pepsin, papain, V8 protease, subtilisin, plasmin, and thrombin. Intact polypeptides can be incubated with one or more proteinases simultaneously or sequentially. Alternatively, or in addition, intact Isd polypeptides can be treated with disulfide reducing agents. Peptides may then be separated from each other by techniques known in the art, including but not limited to, gel filtration chromatography, gel electrophoresis, and reverse-phase HPLC.

6. Isd Antibodies and Uses Thereof

To produce antibodies against IsdA, IsdB, and/or IsdC, host animals may be injected with full-length Isd polypeptides or with Isd polypeptides or peptides. Hosts may be injected with peptides of different lengths encompassing a desired target sequence. For example, peptide antigens that are at least 5, 10, 15, 20, 25, 30, 35, 40, 45, 50, 55, 60, 65, 70, 75, 80, 85, 90, 95, 100, 105, 110, 115, 120, 125, 130, 135, 140, 145 or 150 amino acids may be used. Alternatively, if a portion of an Isd protein defines an epitope, but is too short to be antigenic, it may be conjugated to a carrier molecule in order to produce antibodies. Some suitable carrier molecules include keyhole limpet hemocyanin, Ig sequences, TrpE, and human or bovine serum albumen. Conjugation may be carried out by methods known in the art. One such method is to combine a cysteine residue of the fragments with a cysteine residue on the carrier molecule.

In addition, antibodies to three-dimensional epitopes, i.e., non-linear epitopes, may also be prepared, based on, e.g., crystallographic data of proteins. Antibodies obtained from that injection may be screened against the short antigens of IsdA, IsdB or IsdC. Antibodies prepared against an Isd peptide may be tested for activity against that peptide as well as the full length Isd protein. Antibodies may have affinities of at least about 10⁻⁶M, 10⁻⁷M, 10⁻¹M, 10⁻⁹M, 10⁻¹⁰M, 10⁻¹¹M or 10⁻¹²M toward the Isd peptide and/or the full length Isd protein.

Suitable cells for the DNA sequences and host cells for antibody expression and secretion can be obtained from a number of sources, including the American Type Culture Collection (“Catalogue of Cell Lines and Hybridomas” 5^{th} edition (1985) Rockville, Md., U.S.A.).

Polyclonal and monoclonal antibodies may be produced by methods known in the art. Monoclonal antibodies may be produced by hybridomas prepared using known procedures including the immunological method described by Kohler and Milstein, Nature 1975; 256: 495-7; and Campbell in “Monoclonal Antibody Technology, The Production and Characterization of Rodent and Human Hybridomas” in Burdon et al., Eds. Laboratory Techniques in Biochemistry and Molecular Biology, Volume 13, Elsevier Science Publishers, Amsterdam (1985); as well as by the recombinant DNA method described by Huse et al., Science (1989) 246: 1275-81.

Methods of antibody purification are well known in the art. See, for example, Harlow and Lane (1988) Antibodies: A Laboratory Manual, Cold Spring Harbor Laboratory, N.Y. Purification methods may include salt precipitation (for example, with ammonium sulfate), ion exchange chromatography (for example, on a cationic or anionic exchange column run at neutral pH and eluted with step gradients of increasing ionic strength), gel filtration chromatography (including gel filtration HPLC), and chromatography on affinity resins such as protein A, protein G, hydroxyapatite, and anti-antibody. Antibodies may also be purified on affinity columns according to methods known in the art.

Other embodiments include functional equivalents of antibodies, and include, for example, chimerized, humanized, and single chain antibodies as well as fragments thereof. Methods of producing functional equivalents are disclosed in PCT Application WO 93/21319; European Patent Application No. 239,400; PCT Application WO 89/09622; European Patent Application 388,745; and European Patent Application EP 332,424.

Functional equivalents include polypeptides with amino acid sequences substantially the same as the amino acid sequence of the variable or hypervariable regions of the antibodies of the invention. “Substantially the same” amino acid sequence is defined herein as a sequence with at least 70%, preferably at least about 80%, and more preferably at least 90% homology to another amino acid sequence as determined by the FASTA search method in accordance with Pearson and Lipman, (1988) Proc Natl Acd Sci USA 85: 2444-8.

Chimerized antibodies may have constant regions derived substantially or exclusively from human antibody constant regions and variable regions derived substantially or exclusively from the sequence of the variable region from a mammal other than a human. Humanized antibodies may have constant regions and variable regions other than the complement determining regions (CDRs) derived substantially or exclusively from the corresponding human antibody regions and CDRs derived substantially or exclusively from a mammal other than a human.

Suitable mammals other than a human may include any mammal from which monoclonal antibodies may be made. Suitable examples of mammals other than a human may include, for example, a rabbit, rat, mouse, horse, goat, or primate.

Antibodies to IsdA, IsdB or IsdC may be prepared as described above use as an anti-infective. In other embodiments, antibodies that recognize functional Isd fragments may also be used in random peptide phage display technology (Eidne et al., Biol Reprod. 63(5):1396-402 (2000)). Briefly, fifteen or twelve-mer random peptide phage display libraries can be used to determine peptides that might interact with functional Isd peptides by competitive displacement of Fab fragments of Isd antibodies. For this, fixed S. aureus cells are allowed to adhere to wells in multiwell plates, and immunostaining for IsdA, IsdB or IsdC may then be evaluated in the absence and presence of unique and random peptides expressed by the phage library. Once the competitive peptides are identified by amino acid sequence analysis, increased amounts of peptide can be synthesized and used as alternative molecular antagonists to antibodies directed against functional fragments. Another alternative is to screen small molecule libraries for their ability to competitively displace Fab fragments to functional IsdA, IsdB, or IsdC fragments. Molecular antagonists identified in this manner may be used to neutralize the effect of antibodies generated by an immune response to the Isd polypeptide or polynucleotide vaccine.

In a further embodiment, the antibodies to IsdA, IsdB, or IsdC (whole antibodies or antibody fragments) may be conjugated to a biocompatible material, such as polyethylene glycol molecules (PEG) according to methods well known to persons of skill in the art to increase the antibody's half-life. See for example, U.S. Pat. No. 6,468,532. Functionalized PEG polymers are available, for example, from Nektar Therapeutics. Commercially available PEG derivatives include, but are not limited to, amino-PEG, PEG amino acid esters, PEG-hydrazide, PEG-thiol, PEG-succinate, carboxymethylated PEG, PEG-propionic acid, PEG amino acids, PEG succinimidyl succinate, PEG succinimidyl propionate, succinimidyl ester of carboxymethylated PEG, succinimidyl carbonate of PEG, succinimidyl esters of amino acid PEGs, PEG-oxycarbonylimidazole, PEG-nitrophenyl carbonate, PEG tresylate, PEG-glycidyl ether, PEG-aldehyde, PEG vinylsulfone, PEG-maleimide, PEG-orthopyridyl-disulfide, heterofunctional PEGs, PEG vinyl derivatives, PEG silanes, and PEG phosphohdes. The reaction conditions for coupling these PEG derivatives will vary depending on the polypeptide, the desired degree of PEGylation, and the PEG derivative utilized. Some factors involved in the choice of PEG derivatives include: the desired point of attachment (such as lysine or cysteine R-groups), hydrolytic stability and reactivity of the derivatives, stability, toxicity and antigenicity of the linkage, suitability for analysis, etc.

7. Pharmaceutical Compositions

Purified IsdA, IsdB, or IsdC polypeptides and nucleic acids may be formulated and introduced as a vaccine through oral, intradermal, intramuscular, intraperitoneal, intravenous, subcutaneous, intranasal, intravaginal, and via scarification (i.e., scratching through the top layers of skin, e.g., using a bifurcated needle) or any other standard route of immunization. Isd polypeptides may further be orally delivered as a vaccine by enteric-coated capsules, which will dissolve in the gut and taken up by antigen presenting cells in Peyer's patches. Oral delivery of Isd polypeptides may supplement injections of Isd polypeptides.

Further, S. aureus anti-Isd antibodies, isd antisense nucleic acids and siRNAs, as described herein may be administered by various means, depending on their intended use, as is well known in the art. For example, if such S. aureus antagonist compositions are to be administered orally, they may be formulated as tablets, capsules, granules, powders or syrups. Alternatively, formulations of the present invention may be administered parenterally as injections (intravenous, intramuscular or subcutaneous), drop infusion preparations or suppositories. For application by the ophthalmic mucous membrane route, compositions of the present invention may be formulated as eyedrops or eye ointments. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compositions may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations, which are suitable for vaginal administration also include pessaries, tampons, creams, gels, pastes, foams or spray formulations containing such carriers as are known in the art to be appropriate.

Dosage forms for transdermal administration of a subject composition includes powders, sprays, ointments, pastes, creams, lotions, gels, solutions, patches and inhalants. The active component may be mixed under sterile conditions with a pharmaceutically acceptable carrier, and with any preservatives, buffers, or propellants, which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions.

Ordinarily, an aqueous aerosol is made by formulating an aqueous solution or suspension of a subject composition with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

In addition, Isd based vaccines may be administered parenterally as injections (intravenous, intramuscular or subcutaneous). The vaccine compositions of the present invention may optionally contain one or more adjuvants. Any suitable adjuvant can be used, such as aluminum hydroxide, aluminum phosphate, plant and animal oils, and the like, with the amount of adjuvant depending on the nature of the particular adjuvant employed. In addition, the anti-infective vaccine compositions may also contain at least one stabilizer, such as carbohydrates such as sorbitol, mannitol, starch, sucrose, dextrin, and glucose, as well as proteins such as albumin or casein, and buffers such as alkali metal phosphates and the like. Preferred adjuvants include the SynerVax™ adjuvant.

Pharmaceutical compositions of this invention suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers, which may be employed in the pharmaceutical compositions of the invention, include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

Further, Isd immunogens or Isd antibodies of the present invention may be encapsulated in liposomes and administered via injection Commercially available liposome delivery systems are available from Novavax, Inc. of Rockville, Md., commercially available under the name Novasomes™. These liposomes are specifically formulated for immunogen or antibody delivery. In an embodiment of the invention Novasomes™ containing Isd peptides or antibody molecules bound to the surface of these non-phospholipid positively charged liposomes may be used.

The pharmaceutical compositions described herein may be used to prevent or treat conditions or diseases resulting from S. aureus infections including, but not limited to a furuncle, chronic furunculosis, impetigo, acute osteomyelitis, pneumonia, endocarditis, scalded skin syndrome, toxic shock syndrome, and food poisoning.

8. Exemplary Screening Assays for Inhibitors of Isd

In general, agents or compounds capable of reducing pathogenic virulence by interfering with iron-regulated surface determinants (Isd) can be identified using the instant disclosed assays to screen large libraries of both natural product or synthetic (or semi-synthetic) extracts or chemical libraries. Those skilled in the field of drug discovery and development will understand that the precise source of agents (e.g., test extracts or compounds) is not critical to the screening procedures of the invention. Accordingly, virtually any number of chemical extracts or compounds can be screened using the methods described herein. Examples of such agents, extracts, or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmnaMar, U.S.A. (Cambridge, Mass.). In addition, natural and synthetically produced libraries are produced, if desired, according to methods known in the art, for example, by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-pathogenic activity should be employed whenever possible.

When a crude extract is found to have an anti-pathogenic or anti-virulence activity, or a binding activity, further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogeneous extracts are known in the art. If desired, compounds shown to be useful agents for the treatment of pathogenicity are chemically modified according to methods known in the art.

Potential inhibitors or antagonists of Isd encoded polypeptides may include organic molecules, peptides, peptide mimetics, polypeptides, and antibodies that bind to a nucleic acid sequence or polypeptide of the invention and thereby inhibit or extinguish its activity. Potential antagonists also include small molecules that bind to and occupy the binding site of the polypeptide thereby preventing binding to cellular binding molecules, such that normal biological activity is prevented. Other potential antagonists include antisense molecules.

Further, S. aureus anti-Isd antagonists as identified by the screening assays described herein may be administered by various means, depending on their intended use, as described above.

8.1 Interaction Assays

Purified and recombinant IsdA, IsdB, and IsdC polypeptides may be used to develop assays to screen for agents that bind to an Isd gene product, and disrupt a protein-protein interaction. Potential inhibitors or antagonists of IsdA, IsdB, or IsdC may include small organic molecules, peptides, polypeptides, peptide mimetics, and antibodies that bind to either IsdA, IsdB, or IsdC and thereby reduce or extinguish its activity.

In certain embodiments, an agent may be identified that binds to an Isd polypeptide and inhibits the uptake of iron comprising the steps of (i) contacting the Isd polypeptide with an appropriate interacting molecule in the presence of an agent under conditions permitting the interaction between the Isd polypeptide and the interacting molecule in the absence of an agent, and (ii) determining the level of interaction between the Isd polypeptide and the interacting molecule, wherein a different level of interaction between the Isd polypeptide and the interacting molecule in the presence of the agent relative to the absence of the agent indicate that the agent inhibits the interaction between the Isd polypeptide and the interacting molecule.

In another embodiment, an agent may be identified that disrupts the interaction between an Isd polypeptide and an interacting molecule. In an exemplary binding assay, a reaction mixture may be generated to include at least a biologically active portion of either IsdA, IsdB, or IsdC, an agent(s) of interest, and an appropriate interacting molecule. An exemplary interacting molecule may be a hemoprotein, hemin, transferrin, fibrinogen or fibronectin. In an exemplary embodiment, the agent of interest is an antibody against a particular Isd polypeptide. Binding of an antibody to an Isd polypeptide may inhibit the function of the Isd polypeptide in binding heme or a hemoprotein. Detection and quantification of an interaction of a particular Isd polypeptide with an appropriate interacting molecule provides a means for determining an agent's efficacy at inhibiting the interaction. The efficacy of the agent can be assessed by generating dose response curves from data obtained using various concentrations of the test agent. Moreover, a control assay can also be performed to provide a baseline for comparison. In the control assay, the interaction of a particular Isd polypeptide with an appropriate interacting molecule may be quantitated in the absence of the test agent.

Interaction between a particular Isd polypeptide and an appropriate interacting molecule may be detected by a variety of techniques. Modulation of the formation of complexes can be quantitated using, for example, detectably labeled proteins such as radiolabeled, fluorescently labeled, or enzymatically labeled polypeptides, by immunoassay, or by chromatographic detection.

The measurement of the interaction of a particular Isd protein with the appropriate interacting molecule may be observed directly using surface plasmon resonance technology in optical biosensor devices. This method is particularly useful for measuring interactions with larger (>5 kDa) polypeptides and can be adapted to screen for inhibitors of the protein-protein interaction.

Alternatively, it will be desirable to immobilize a particular Isd polypeptide or the appropriate interacting molecule to facilitate separation of complexes from uncomplexed forms of one or both of the proteins, as well as to accommodate automation of the assay. Binding of a particular Isd protein to the interacting molecule for example, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/IsdA (GST/IsdA) fusion proteins can be adsorbed onto glutathione sepharose beads (Sigma Chemical, St. Louis, Mo.) or glutathione derivatized microtitre plates, which are then combined with, for example, an ³⁵S-labeled interacting molecule, and the test agent, and the mixture incubated under conditions conducive to complex formation, for example, at physiological conditions for salt and pH, though slightly more stringent conditions may be desired. Following incubation, the beads are washed to remove any unbound label, and the matrix immobilized and radiolabel determined directly (e.g., beads placed in scintillant), or in the supernatant after the complexes are subsequently dissociated. Alternatively, the complexes can be dissociated from the matrix, separated by SDS-PAGE, and the level of interacting molecule found in the bead fraction quantitated from the gel using standard electrophoretic techniques.

Other techniques for immobilizing proteins and other molecules on matrices are also available for use in the subject assay. For instance, either a particular Isd protein or the appropriate interacting molecule can be immobilized utilizing conjugation of biotin and streptavidin. For instance, biotinylated IsdA, IsdB, or IsdC can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with either IsdA, IsdB, or IsdC but which do not interfere with the interaction between the polypeptide and the interacting molecule, can be derivatized to the wells of the plate, and IsdA, IsdB, or IsdC may be trapped in the wells by antibody conjugation. As above, preparations of an interacting molecule and a test compound may be incubated in the polypeptide-presenting wells of the plate, and the amount of complex trapped in the well can be quantitated in the presence or absence of a test agent. Exemplary methods for detecting such complexes, in addition to those described above for the GST-immobilized complexes, include immunodetection of complexes using antibodies reactive with the interacting molecule or enzyme-linked assays, which rely on detecting an enzymatic activity associated with the interacting molecule.

For example, an enzyme can be chemically conjugated or provided as a fusion protein with the interacting molecule. To illustrate, the interacting molecule can be chemically cross-linked or genetically fused with horseradish peroxidase, and the amount of polypeptide trapped in the complex can be assessed with a chromogenic substrate of the enzyme, for example, 3,3′-diamino-benzadine terahydrochloride or 4-chloro-1-napthol. Likewise, a fusion protein comprising the polypeptide and glutathione-S-transferase can be provided, and complex formation quantitated by detecting the GST activity using 1-chloro-2,4-dinitrobenzene (Habig et al. (1974) J. Biol. Chem. 249:7130).

8.2 Expression Assays

In a further embodiment, antagonists of iron uptake may affect the expression of isdA, isdB, and isdC nucleic acid or protein. In this screen, S. aureus cells may be treated with a compound(s) of interest, and then assayed for the effect of the compound(s) on isdA, isdB, and isdC nucleic acid or protein expression.

In certain embodiments, an agent maybe identified that inhibits the expression of an Isd polypeptide in Staphylococcus aureus comprising the step of (i) culturing a wild type Staphylococcus aureus strain in the presence or absence of said agent; and (ii) comparing the expression of Isd polypeptides wherein a greater reduction in the expression of Isd polypeptides in cells treated with said agent indicates that said agent inhibits the expression of Isd polypeptides in Staphylococcus aureus.

In an alternate embodiment, an agent may be identified that inhibits the expression of an isd nucleic acid in Staphylococcus aureus comprising the step of (i) culturing a wild type Staphylococcus aureus strain in the presence or absence of said agent; and (ii) comparing the expression of isd nucleic acids wherein a greater reduction in the expression of isd nucleic acids in cells treated with said agent indicates that said agent inhibits the expression of isd nucleic acids in Staphylococcus aureus.

For example, total RNA can be isolated from S. aureus cells cultured in the presence or absence of test agents, using any suitable technique such as the single-step guanidinium-thiocyanate-phenol-chloroform method described in Chomczynski et al. (1987) Anal. Biochem. 162:156-159. The expression of isdA, isdB, or isdC may then be assayed by any appropriate method such as Northern blot analysis, the polymerase chain reaction (PCR), reverse transcription in combination with the polymerase chain reaction (RT-PCR), and reverse transcription in combination with the ligase chain reaction (RT-LCR). Northern blot analysis can be performed as described in Harada et al. (1990) Cell 63:303-312. Briefly, total RNA is prepared from S. aureus cells cultured in the presence of a test agent. For the Northern blot, the RNA is denatured in an appropriate buffer (such as glyoxal/dimethyl sulfoxide/sodium phosphate buffer), subjected to agarose gel electrophoresis, and transferred onto a nitrocellulose filter. After the RNAs have been linked to the filter by a UV linker, the filter is prehybridized in a solution containing formamide, SSC, Denhardt's solution, denatured salmon sperm, SDS, and sodium phosphate buffer. A S. aureus isdA, isdB, or isdC DNA sequence may be labeled according to any appropriate method (such as the ³²P-multiprimed DNA labeling system (Amersham)) and used as probe. After hybridization overnight, the filter is washed and exposed to x-ray film. Moreover, a control can also be performed to provide a baseline for comparison. In the control, the expression of isdA, isdB, or isdC in S. aureus may be quantitated in the absence of the test agent.

Alternatively, the levels of mRNA encoding IsdA, IsdB, and IsdC polypeptides may also be assayed, for e.g., using the RT-PCR method described in Makino et al. (1990) Technique 2:295-301. Briefly, this method involves adding total RNA isolated from S. aureus cells cultured in the presence of a test agent, in a reaction mixture containing a RT primer and appropriate buffer. After incubating for primer annealing, the mixture can be supplemented with a RT buffer, dNTPs, DTT, RNase inhibitor and reverse transcriptase. After incubation to achieve reverse transcription of the RNA, the RT products are then subject to PCR using labeled primers. Alternatively, rather than labeling the primers, a labeled dNTP can be included in the PCR reaction mixture. PCR amplification can be performed in a DNA thermal cycler according to conventional techniques. After a suitable number of rounds to achieve amplification, the PCR reaction mixture is electrophoresed on a polyacrylamide gel. After drying the gel, the radioactivity of the appropriate bands may be quantified using an imaging analyzer. RT and PCR reaction ingredients and conditions, reagent and gel concentrations, and labeling methods are well known in the art. Variations on the RT-PCR method will be apparent to the skilled artisan. Other PCR methods that can detect the nucleic acid of the present invention can be found in PCR Primer: A Laboratory Manual (Dieffenbach et al. eds., Cold Spring Harbor Lab Press, 1995). A control can also be performed to provide a baseline for comparison. In the control, the expression of isdA, isdB, or isdC in S. aureus may be quantitated in the absence of the test agent.

Alternatively, the expression of IsdA, IsdB, and IsdC polypeptides may be quantitated following the treatment of S. aureus cells with a test agent using antibody-based methods such as immunoassays. Any suitable immunoassay can be used, including, without limitation, competitive and non-competitive assay systems using techniques such as western blots, radioimmunoassays, ELISA (enzyme-linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

For example, IsdA, IsdB, or IsdC polypeptides can be detected in a sample obtained from S. aureus cells treated with a test agent, by means of a two-step sandwich assay. In the first step, a capture reagent (e.g., either a IsdA, IsdB, or IsdC antibody) is used to capture the specific polypeptide. The capture reagent can optionally be immobilized on a solid phase. In the second step, a directly or indirectly labeled detection reagent is used to detect the captured marker. In one embodiment, the detection reagent is an antibody. The amount of IsdA, IsdB, or IsdC, polypeptide present in S. aureus cells treated with a test agent can be calculated by reference to the amount present in untreated S. aureus cells.

Suitable enzyme labels include, for example, those from the oxidase group, which catalyze the production of hydrogen peroxide by reacting with substrate. Glucose oxidase is particularly preferred as it has good stability and its substrate (glucose) is readily available. Activity of an oxidase label may be assayed by measuring the concentration of hydrogen peroxide formed by the enzyme-labeled antibody/substrate reaction. Besides enzymes, other suitable labels include radioisotopes, such as iodine (¹²⁵I, ¹²¹I), carbon (¹⁴C), sulphur (³⁵S), tritium (³H).

Examples of suitable fluorescent labels include a fluorescein label, an isothiocyanate label, a rhodamine label, a phycoerythrin label, a phycocyanin label, an allophycocyanin label, an o-phthaldehyde label, and a fluorescamine label.

Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase. Examples of chemiluminescent labels include a luminol label, an isoluminol label, an aromatic acridinium ester label, an imidazole label, an acridinium salt label, an oxalate ester label, a luciferin label, a luciferase label, and an aequorin label.

Exemplification

The invention, having been generally described, may be more readily understood by reference to the following examples, which are included merely for purposes of illustration of certain aspects and embodiments of the present invention, and are not intended to limit the invention in any way.

### EXAMPLE 1

### Expression of IsdA, IsdB and IsdC Proteins

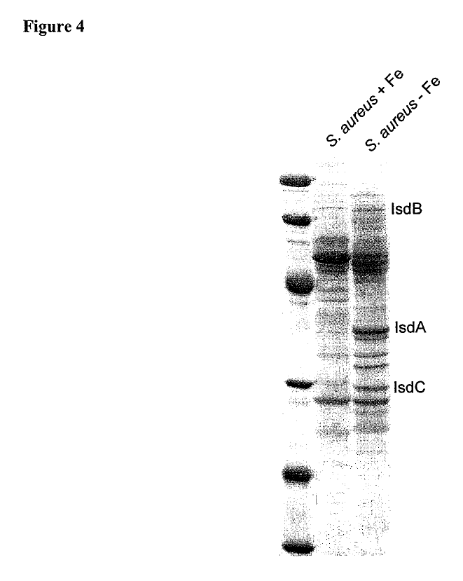

IsdA, IsdB, and IsdC proteins are expressed under iron-limiting conditions as shown in FIG. 4 (S. aureus−Fe). The SDS-PAGE gel shown in FIG. 4 illustrates that the IsdA, IsdB, and IsdC proteins are three of the most predominant iron regulated proteins expressed by S. aureus. These proteins are not expressed when the S. aureus cells are cultured in iron-rich media (S. aureus+Fe) and are, therefore, by inference likely all highly expressed in vivo.

Overexpression of IsdA, IsdB, and IsdC, as well as IsdE, as fusions in E. coli results in highly colored lysates. Absorptions and magnetic circular dichroism spectroscopy was used to confirm that this coloration was due to the ability of the proteins to scavenge different forms of protoporphyrin and heme from within the E. coli cytoplasm, confirming their role in heme binding.

### EXAMPLE 2

### Generation of isd Gene Knockout Mutants

Further, the coding regions of isdA, isdB and isdC were interrupted individually to generate strains that contain a single mutation in each of the isd genes. The isdA coding region was interrupted by inserting a cassette encoding resistance to tetracycline. The isdB coding region was interrupted by inserting a cassette encoding resistance to erythromycin. The isdC coding region was interrupted by inserting a cassette encoding resistance to kanamycin. Each mutation was then moved into the same genetic background using phage transduction procedures and selected for using the appropriate resistance as described in Sebulsky et al., (2001) J. Bacteriol. 183:4994-5000. Further, strains containing mutations knocking out two or more of the isd genes (e.g., a strain mutated in isdA, isdB, and isdC may also be generated.

### EXAMPLE 3

### Survival Studies in the Mouse Model of Kidney Infection

Female Swiss-Webster mice, weighing 25 g, were purchased from Charles River Laboratories Canada, Inc., and housed in microisolator cages. Bacteria were grown overnight in Tryptic Soy Broth (TSB), harvested and washed three times in sterile saline. Pilot experiments demonstrated that S. aureus Newman colonized mice better in this model than did RN6390, and that the optimal amount of S. aureus Newman to inject into the tail vein to obtain an acute, but non-lethal kidney infection was 1×10⁷ CFU. Bacteria, suspended in sterile saline, were administered intravenously via the tail vein. The number of viable bacteria injected were confirmed by plating serial dilutions of the inoculum on TSB. On day six post-injection, mice were sacrificed and kidneys were aseptically removed. Using a PowerGen 700 Homogenizer, kidneys were homogenized for 45 seconds in sterile PBS containing 0.1% Triton X-100 and homogenate dilutions were plated on TSB-agar to enumerate viable bacteria. Data presented are the log CFU recovered per mouse.

Results indicate that mutations in either IsdA alone, or in a strain carrying mutations in all of IsdA, IsdB, and IsdC attenuate S. aureus virulence using a murine kidney abscess model of S. aureus infection. Interestingly, after 6 days post-infection, recovered mutant bacteria are 90% decreased from the numbers recovered from the wildtype, thus indicating that these proteins, when expressed on the bacterial cell surface, play a essential role in the fitness of the bacteria during infection. This also indicates then that inhibition of these proteins in vivo could either prevent infection by Isd-expressing bacteria (i.e., in the case of an Isd-based vaccine) or could result in clearance of the Isd-expressing bacteria once infection was initiated.

### EXAMPLE 4

### Survival of S. aureus Under Increasing Hydrogen Peroxide Concentration

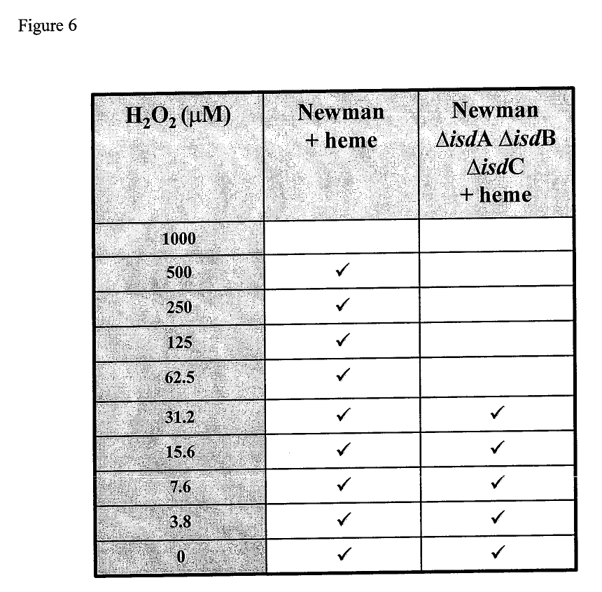

Isd proteins bound to heme appear to act as an oxidative buffer that protects S. aureus cells from the detrimental effects of free radicals. A direct comparison of Newman strains incubated in the presence of heme to Newman strains deleted for IsdA, IsdB, and IsdC incubated in the presence of heme shows that mutant cells were not able to survive increased concentrations of hydrogen peroxide (FIG. 6). Thus, mutants lacking the expression of several Isd proteins are more susceptible to challenge with hydrogen peroxide.

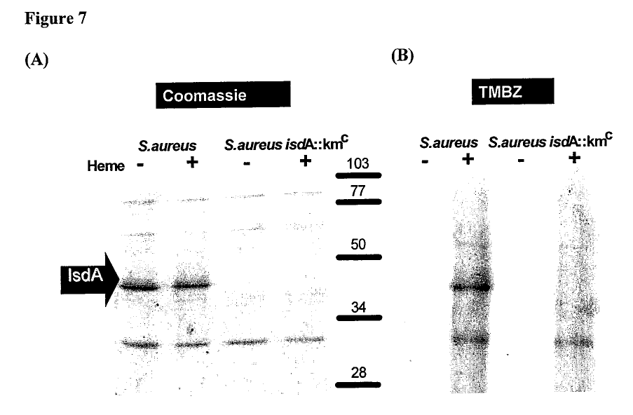

FIG. 7 shows the expression of IsdA plus and minus heme in both wild type S. aureus and S. aureus isdA::km^{c} run on an SDS-PAGE gel stained with (A) Coomassie and (B) TMBZ (tetramethylbenzidine). Catalase activity associated with the heme-bound form of IsdA cleaves the TMBZ compound to yield a colored reaction product. Thus, heme-bound IsdA has catalase activity that may help resist the oxidative killing by phagocytes.

### EXAMPLE 5

### Isd Vaccines

A vaccine comprising recombinant IsdA polypeptide can establish protective immunity in mice against systemic and localized S. aureus infection. Recombinant IsdA protein may be prepared using standard techniques. Groups of 12-15 Swiss-Webster mice (25 g) can be used for all immunization experiments and injected intraperitoneally (IP). Mice can be boosted with subsequent injections at various different time points. Sera can be monitored over the course of the experiment for anti-IsdA antibody titres. On approximately day 30, mice can be challenged intravenously with 1×10⁷ S. aureus and monitored for a further 7 days. We have previously shown that injection with this number of live organisms results in non-fatal kidney infections. Mice can be sacrificed at various time points post infection to monitor the number of organisms infecting the kidney tissue. Passive immunization experiments can also be performed using sera collected from previously immunized mice to examine their effectiveness at preventing infection in other groups of mice. Similar immunization experiments can be conducted with IsdB and IsdC polypeptides.

### EXAMPLE 6

### IsdA, IsdB, and IsdC Antibodies

A. Preparation of Monoclonal Antibodies Against Full-Length Isd Proteins

BALB/c mice can be immunized initially via intraperitoneal injections with full-length recombinant IsdA, IsdB, or IsdC and later boosted similarly with native IsdA, IsdB, or IsdC approximately six weeks later. The mice can be immunized with an appropriate adjuvant. Mouse serum can be obtained approximately ten days after the second injection and then tested for anti-HRP activity via ELISA. The mice whose serum exhibits high levels of anti-HRP activity can be chosen for cell fusion. Spleens can be collected from these mice and cell suspensions prepared by perfusion with Dulbecco's Modified Eagle Medium (DMEM).

Spleen cell suspension containing B-lymphocytes and macrophages can be prepared by perfusion of the spleen. The cell suspension can be washed and collected by centrifugation; myeloma cells can also be washed in this manner. Live cells can be counted and the cells can be placed into a 37° C. water bath. One mL of 50% polyethylene glycol (PEG) can be added to DMEM. The Balb/c spleen cells can be fused with SP 2/0-Ag 14 mouse myeloma cells by PEG and the resultant hybridomas can be grown in hypoxanthine (H), aminopterin (A) and thymidine (T) (HAT) selected tissue culture media plus 20% fetal calf serum. The surviving cells can be allowed to grow to confluence. The spent culture medium can be checked for antibody titer, specificity, and affinity. The cells can be incubated in the PEG for one to 1.5 minutes at 37° C., after which the PEG was diluted by the slow addition of DMEM media. The cells can be pelleted and 35 to 40 mL of DMEM containing 10% fetal bovine serum may be added. The cells can then be dispensed into tissue culture plates and incubated overnight in a 37° C., 5% CO₂, humidified incubator.

The next day, DMEM-FCS containing hypoxanthine (H), aminopterin (A) and thymidine (T) medium (HAT medium) can be added to each well. The concentration of HAT in the medium to be added can be twice the final concentration required, i.e., H_{final}=1 times 10⁻⁴M; A_{final}=4 times 10⁻⁷M; and T_{final}=1.6 times 10⁻⁵M.

Subsequently, the plates can be incubated with HAT medium every three to four days for two weeks. Fused cells can be then cultured in DMEM-FCS containing HAT medium. As fused cells become ½ to ¾ confluent on the bottom of the wells, supernatant tissue culture fluid can be taken and tested for IsdA, IsdB, or IsdC specific antibodies by ELISA. Positive wells can be cloned by limiting dilution over macrophage or thymocyte feeder plates, and cultured in DMEM-FCS. Cloned wells can be tested and recloned three times before a statistically significant monoclonal antibody can be obtained. Spent culture media can be tested from the antibody-producing clones.

B. Preparation of Polyclonal Antibodies Against Isd Proteins

Unconjugated purified recombinant IsdA, IsdB and/of IsdC or portions thereof can be used as antigens to immunize rabbits. Briefly, 1 mg of the antigen can be resuspended in 1 ml of phosphate buffered saline and emulsified with an equal volume of Complete Freund's Adjuvant and approximately 1 ml (half of the total volume) can be injected into each rabbit intraperitoneally. A second and third immunization can follow two and three weeks later, using Incomplete Freund's Adjuvant. Sera may be tested using enzyme-linked inmunosorbent assays (ELISA) to determine specific antibody titers. Sera that exhibits high titer based on ELISA results can be purified by affinity chromatography on a Sepharose column conjugated with corresponding recombinant Isd polypeptide and immunoglobulins can be tested for the ability to attenuate the virulence of S. aureus infection.

### EXAMPLE 7

### Expression Assays

Assays to screen for agents that disrupt the expression of IsdA in S. aureus can be conducted as follows. Wild type S. aureus cells can be cultured overnight in tryptic soy broth (TSB) (Difco) in the presence or absence of a test agent. Following 24 hours of culture, the cells can be washed in 1× PBS (phosphate buffered saline) and then lysed at 37° C. using 10 μg of lysostaphin in STE (0.1 M NaCl, 10 mM Tris-HCl [pH 8.0], 1 mM EDTA [pH 8.0]). The cell lysates can then be transferred to anti-IsdA antibody precoated plates and incubated for 45 to 60 minutes at room temperature. As a control, cell lysates from untreated S. aureus cells can be used. After three washes with water, a secondary antibody conjugated to either alkaline phosphatase (AP) or horseradish peroxidase (HRP) can be added and incubated for one hour. The plate can then be washed to separate the bound from the free antibody complex. A chemiluminescent substrate (alkaline phosphatase or Super Signal luminol solution from Pierce for horseradish peroxidase) can be used to detect bound antibody. A microplate luminometer can be used to detect the chemiluminescent signal. The absence of the signal in samples of cell lysates obtained from cells treated with test agent may indicate that the test agent inhibits the expression of IsdA. Similar expression assays may also be conducted for IsdB and IsdC.

### EXAMPLE 8

### Acquisition of Heme-Iron by S. aureus is Enhanced by IsdA

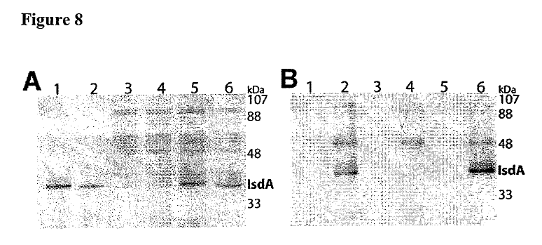

To assess the affect of isdA on the biology of S. aureus with respect to the ability of this bacterium to utilize heme as a sole source of iron, the isdA gene was insertionally inactivated with a tetracycline resistance cassette in the chromosome of S. aureus Newman to create strain H734. When cell wall protein fractions were taken from S. aureus cultures and stained for peroxidase activity to identify heme proteins, wild type cells (strain Newman) incubated with heme show clear staining of a band corresponding to IsdA that is not present in H734 (isdA-) cells (FIG. 8). This band is restored in H734 containing plasmid pJT35 which expresses a cloned isdA gene (FIG. 8).

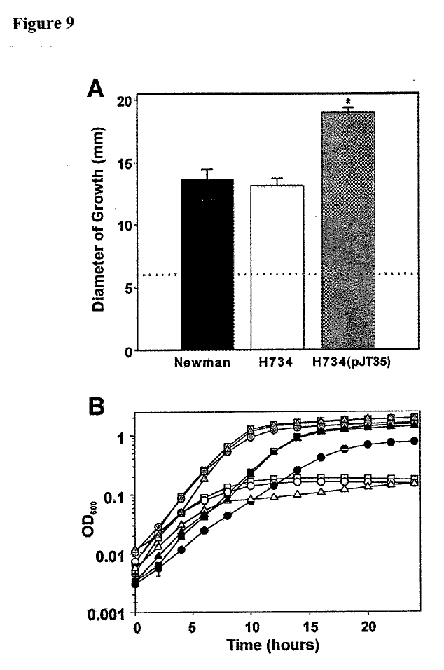

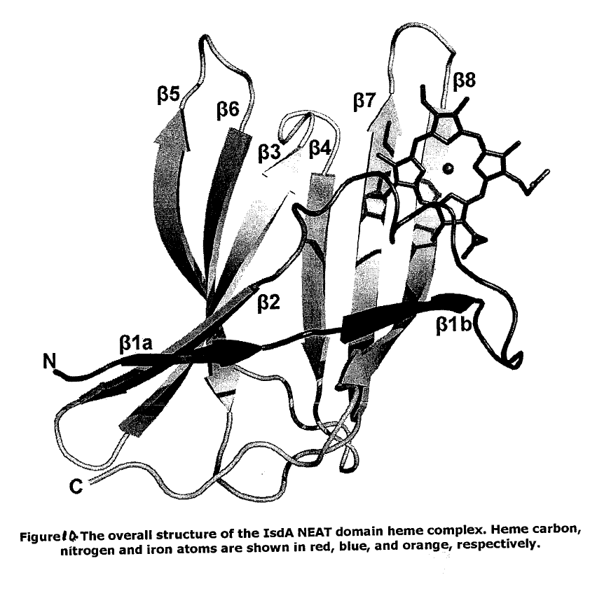

Two different bioassays were performed to assess the significance of IsdA in the acquisition of heme as a source of iron. In a plate assay, there was no observable growth defect in H734 relative to Newman (FIG. 9A). Notably, however, H734 expressing isdA from plasmid pJT35 showed a 40% growth enhancement on heme as a sole source of iron. In an alternative liquid culture assay, we demonstrated that while there was no difference in growth rate or yield between strain Newman and strain H734 (Newman isdA::tet) in iron-replete or iron-restricted chemically defined media (TMS), there was a significant difference in the growth rate and yield between the two strains when heme was provided as a sole source of iron (FIG. 9B). This effect is due to the specific lack of isdA expression in the H734 strain because introduction of pJT35 (which contains only isdA) into H734 complemented the growth defect (FIG. 9B).

### EXAMPLE 9

### The IsdA NEAT Domain Binds Heme

We expressed only the IsdA NEAT domain in E. coli and found that it alone was capable of binding heme as judged by UV-visible spectroscopy at 280 and 407 nm. An absorbance ratio of 280 nm to 407 nm indicated that the NEAT domain, as expressed from E. coli, was approximately ⅔ saturated with heme. Partial heme occupancy is typical for heme transport proteins expressed intracellularly in E. coli (Eakanunkul et al., 2005; Mack et al., 2003; Schneider et al., 2006). To ensure sample homogeneity for protein crystallization, apo and holo protein were separated by anion exchange chromatography. The apo protein was then crystallized as purified and after reconstitution with heme. Reconstituted IsdA resembles the holo protein fraction as purified from E. coli, except for a greater ratio of the heme Soret (A₄₀₇) to A₂₈₀ absorption, indicating greater substrate saturation.

### EXAMPLE 10

### The IsdA NEAT Domain Structure Reveals Heme-Iron Coordination by a Conserved Tyrosine

The IsdA NEAT domain apo-structure was solved to 1.6 Å resolution by seleno-methionine labelling and multiwavelength anomalous dispersion phasing (Hendrickson, 1991), whereas the IsdA NEAT holo-structure was solved to 1.9 Å resolution by molecular replacement. The IsdA NEAT domain forms an Ig-like fold composed of seven β-stands forming two sheets of a β-sandwich. However, an additional eighth β-strand is present which results in the N— and C-termini being located in close proximity (FIG. 10). The recently-described fold of the IsdH/HarA NEAT domain 1 (19% sequence identity to the IsdA NEAT domain) is similar except for residues forming an additional N-terminal strand on one β-sheet such that the N and C-termini are on opposite ends of the molecule (Pilpa et al., 2006). Although no significant sequence similarity is present, a Dali search (Holm and Sander, 1993) reveals that the IsdA NEAT domain fold is similar to that of the clathrin adapter appendage superfamily (2.9 Å r.m.s.d. for 94 aligned Ca atoms to PDB entry 1GYU). The NEAT domain fold is unique with respect to heme binding proteins.

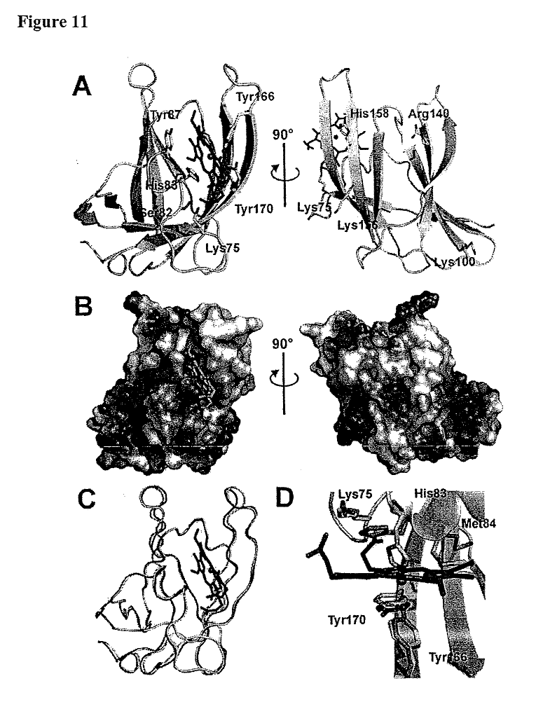

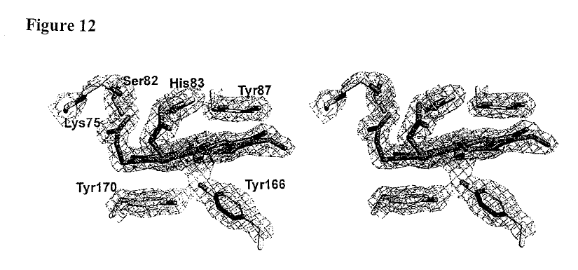

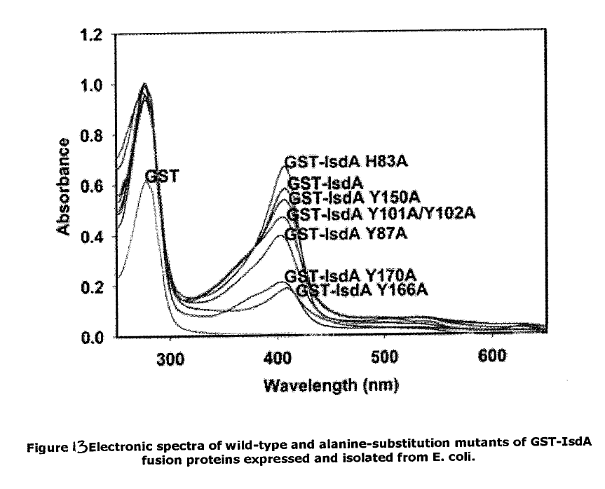

The NEAT domain β-sandwich is splayed open at the end opposite the chain termini, forming a pocket between one of the β-sheets and a loop formed from residues Ser⁸² to Tyr⁸⁷ (FIG. 11A). Heme binding in this pocket does not cause significant displacement of the backbone (0.53 Å r.m.s.d) or side-chain atoms of most residues in the IsdA NEAT domain (FIG. 11C). Only His⁸³ and Met⁸⁴ appear to undergo significant conformational changes (FIG. 11D). The side-chain of His⁸³ rotates ˜135° about χ₁ to vacate the pocket and form a hydrogen bond to one of the heme propionates. Met⁸⁴ also shifts to accommodate the heme by moving deeper into the binding pocket (SD displacement of ˜4.7 Å). The heme group is bound in the hydrophobic pocket in two equally occupied orientations related to one another by a 180° rotation about the α,γ-meso axis. In both orientations the propionate groups overlap and are facing out towards the solvent. Approximately 278 Å² (˜35%) of heme surface area is exposed to solvent (as determined with AREAIMOL (Collaborative Computational Project, 1994)). The propionates form hydrogen bonds with residues Lys⁷⁵ (2.86 Å), Ser⁸² (2.62 Å) and His⁸³ (2.87 Å) (FIG. 11A and FIG. 12).

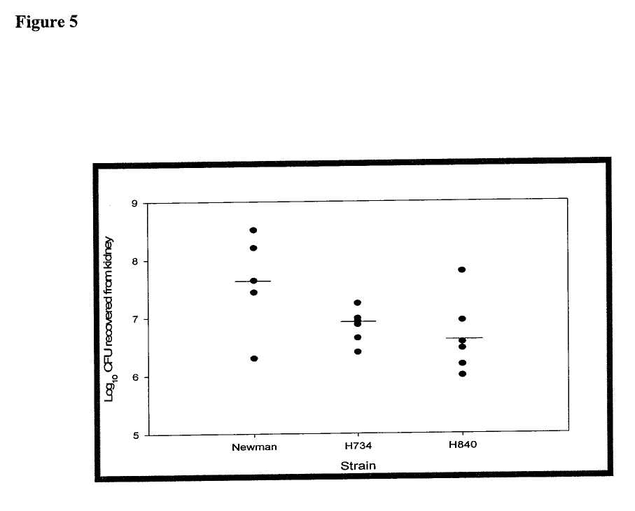

The crystal structure of the NEAT domain-heme complex reveals that the iron is five-coordinate, with the axial ligand (2.11 Å) provided by the phenolic oxygen of conserved Tyr¹⁶⁶ (FIGS. 4A and 5). The iron atom is displaced from the heme nitrogen plane towards the coordinating oxygen by 0.35 Å and the angle between the phenol ring and the ligand bond is ˜130°. Tyr¹⁶⁶, in turn, forms a hydrogen bond (2.55 Å) to Tyr¹⁷⁰ OH (FIG. 5). His⁸³ is positioned on the opposite side of the tetrapyrrole plane to Tyr¹⁷⁰; however, the imidazole ring is coplanar to the heme and does not serve as a second axial ligand. Several hydrophobic residues that comprise the binding pocket, namely Met⁸⁴, Tyr⁸⁷, Phe¹¹², Trp¹¹³, Va¹⁵⁷, Ile¹⁵⁹, Val¹⁶¹ and Tyr¹⁷⁰ contact the tetrapyrrole rings of the heme.

### EXAMPLE 11

### IsdA Point Mutants Show that Tyr¹⁶⁶ and Tyr¹⁷⁰ are Essential for Heme Binding

Spectroscopic studies of full length (excluding signal peptide and C-terminal sorting sequences) IsdA reveal a strong absorbance at 407 nm (A₄₀₇/A₂₈₀ ratio=0.629), as well as signals in the visible region at 503 nm, 538 nm and 625 nm, indicative of heme binding (FIG. 13). Alanine scanning mutagenesis of GST-IsdA was performed to monitor the significance of IsdA residues for heme binding (FIG. 13). Mutation of the conserved heme-iron coordinating residue, Tyr¹⁶⁶, to alanine resulted in almost complete abolition of absorption at 407 nm (A₄₀₇/A₂₈₀ ratio=0.20) indicating that this residue is essential for heme binding. Mutation of Tyr¹⁷⁰ to Ala (A₄₀₇/A₂₈₀ ratio=0.23) also significantly affects heme binding while mutation of Tyr⁸⁷ to Ala (A₄₀₇/A₂₈₀ ratio=0.40) diminishes, but does not abolish, heme binding. These latter two residues are situated in the heme binding pocket and provide hydrophobic interactions in addition to the H-bond between Tyr¹⁷⁰ and Tyr¹⁶⁶. That His⁸³ is not involved in coordinating heme-iron is aptly demonstrated by the result that a His⁸³ to Ala mutation does not at all diminish heme binding (A₄₀₇/A₂₈₀ ratio=0.68). Since magnetic circular dichroism of IsdA identified a tyrosyl residue as the axial ligand (Vermeiren et al., 2006), alanine substitution of all remaining NEAT domain tyrosines (Tyr¹⁰¹, Tyr¹⁰² and Tyr¹⁵⁰) was performed. None of these tyrosines are found within the heme binding pocket in the crystal structure and none of these substitutions affected the absorbance spectra of IsdA compared to wild type protein.

### EXAMPLE 12

### NEAT Domain Sequence-Structure Alignments Reveal that the Tyrosine Ligand is a Prognosticator of Heme Binding

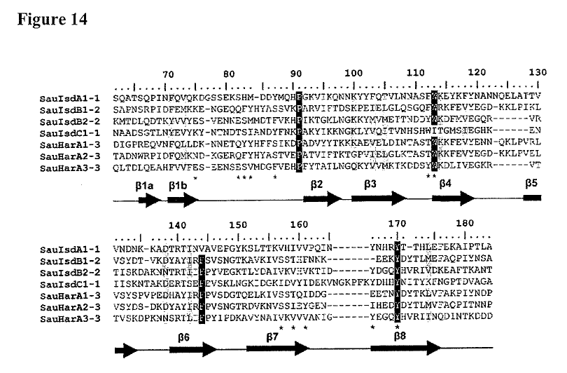

Recognition of heme is by a deep, largely hydrophobic pocket formed by a single IsdA NEAT domain. A multiple sequence alignment of a representative set of 43 NEAT domains is given in FIG. S1 and the seven NEAT domains found in S. aureus (one in IsdA, two in IsdB, one in IsdC, and three in IsdH/HarA) are presented in FIG. 14. Three residues in IsdA that make key heme interactions, Ser⁸², Tyr¹⁶⁶, and Tyr¹⁷⁰, are generally conserved in a large number of NEAT domains (FIG. S1) but are present in only four out of the seven S. aureus NEAT domains (FIG. 14). Furthermore, the residues that form the hydrophobic heme binding pocket in IsdA are either conserved or replaced with similar hydrophobic amino acids in most NEAT domains. In S. aureus NEAT domains with a Tyr residues aligned at positions 166 and 170, heme pocket residues at positions 157, 159 and 161 are either valine or isoleucine and residue 87 is either tyrosine or phenylalanine (FIG. 14). Pilpa et al. identified conserved tyrosine and histidine residues (potential heme-iron ligands) in S. aureus NEAT domains known to bind heme that were absent in those NEAT domains shown not to bind heme, such as the N-terminal NEAT domain in IsdH/HarA (Pilpa et al., 2006). Thus, Tyr⁵², Tyr¹³², His¹³⁴ and Tyr¹³⁶ in IsdC, which correspond to Tyr⁸⁷, Tyr¹⁶⁶, His¹⁶⁸ and Tyr¹⁷⁰ in IsdA, were speculated to be potential heme-iron ligands (Pilpa et al., 2006). Now, based upon a distinct pattern of conservation of amino acid residues observed to interact with the heme in the IsdA structure, we predict that the single NEAT domain of IsdC, and the C-terminal NEAT domains of IsdB and IsdH/HarA bind heme and coordinate the heme-iron by the residue analogous to Tyr¹⁶⁶ in IsdA. The other S. aureus NEAT domains either do not bind heme, or bind heme in a completely different manner.

### EXAMPLE 13

### Discussion of NEAT Domain

The surface potential of the IsdA NEAT domain reveals two large electropositive regions, one located near the polypeptide chain termini, and the other on the same face of the molecule, behind the heme binding pocket (FIG. 11B). Alignments reveal several conserved residues, Lys⁷⁵, Lys¹⁰⁰, Arg¹⁴⁰, Lys¹⁵⁶ and His¹⁵⁸ (see FIG. S1), contributing to these positively charged regions (FIG. 11A). Interestingly, hemopexin, a proposed IsdA substrate (Skaar and Schneewind, 2004), has two dominant negatively charged surfaces near the tunnel entrance of each β-propeller domain (Paoli et al., 1999). The location of the complementary charge surfaces on hemopexin and IsdA, should such an interaction occur, suggests that host protein binding and heme transfer may require more than one IsdA molecule. Non-specific, electrostatic interactions may help to explain the ability of IsdA to interact with several different host proteins (Clarke et al., 2004). Alternatively, the positive surfaces on the IsdA NEAT domain may assist in the orientation of the protein with respect to the negatively charged cell wall or provide an interaction surface for other components of the Isd heme uptake system that convey the heme to the membrane transport complex. In contrast, analysis of the solution structure of the IsdH/HarA N-terminal NEAT domain reveals a large surface with a negative potential (Pilpa et al., 2006). This surface is suggested to be involved in the binding of hemoglobin by IsdH/HarA in a 2:1 stoichiometry (Pilpa et al., 2006).

Heme-iron bound by a single axial tyrosine ligand is uncommon in heme proteins but is an emerging characteristic of heme transporters, including serum albumin, ShuT from Shigella dysenteriae and ChaN from Campylobacter jejuni (Chan et al., 2006; Eakanunkul et al., 2005; Zunszain et al., 2003). The binding of heme to the hemophore from Serratia marcescens, HasA, and the heme chaperone, CcmE, involved in cytochrome c maturation are also similar to IsdA; however, in these instances, an additional histidine residue is coordinated to the iron (Amoux et al., 1999; Uchida et al., 2004). Similar to the IsdA NEAT domain, the multifunctional catalases coordinate heme by a single tyrosine. Moreover, and also like the IsdA NEAT domain, a histidine is present on the opposite side of heme in catalase, but does not coordinate to the iron (Putnam et al., 2000). No catalase activity could be detected for the IsdA NEAT domain and this is likely because His⁸³ blocks the sixth coordination position of the heme iron (FIG. 12).

That His⁸³ is not a ligand to heme-iron in the IsdA crystal structure, as observed in HasA and CcmE, is further supported by the fact that heme binding is not diminished in the His⁸³ to Ala mutant (FIG. 13). In fact, the observed slight increase in heme loading of the mutant relative to wild type IsdA, might be explained by a less constricted heme binding pocket in the mutant protein. Moreover, histidine is not found aligned at position 83 in any other NEAT domain from S. aureus, nor is this position conserved among NEAT domains in general (FIG. S1 and FIG. 14).

In contrast, Tyr¹⁷⁰ is generally conserved in NEAT domain sequences. Tyr¹⁷⁰ may play a similar role to His⁸³ in HasA, where it forms a hydrogen bond to the heme-iron coordinating Tyr⁷⁵ (Arnoux et al., 1999). In HasA, protonation of His83 is proposed to alter the affinity of Tyr⁷⁵ for heme (Arnoux et al., 1999). Similarly, the hydrogen bond observed between Tyr¹⁷⁰ and Tyr¹⁶⁶ in the IsdA NEAT domain crystal structure may control heme affinity by altering the pKa of the pheonolate iron ligand. Indeed, the Tyr¹⁷⁰ to Ala IsdA mutant protein exhibited reduced heme loading as isolated from E. coli (FIG. 13).

In general, bound heme is more solvent exposed in heme transport proteins than is typical of non-transport heme proteins. For heme bound to IsdA, 35% of the surface area is exposed to the solvent. The solvent exposure of heme atoms observed in the structures of HasA (Arnoux et al., 1999), ChaN (Chan et al., 2006), HemS (Schneider et al., 2006) and hemopexin (Paoli et al., 1999) ranges from 18% to 26%. In contrast, the exposure to solvent of heme cofactors of enzymes such as myoglobin and cytochrome c is typically less than 15%. Exposure to a high dielectric solvent such as water is correlated with a lowering of the reduction potential of heme (Mauk and Moore, 1997) and the reduction potential of HasA is unusually low (−550 mv) (Izadi et al., 1997). Therefore, high solvent exposure of the heme group in heme transport proteins likely aids in stabilization of the ferric (Fe³⁺) state of heme iron. The ferric oxidation state of heme is less reactive with oxygen and may facilitate release of the heme group.

That the N-terminal NEAT domain in IsdH/HarA does not bind heme (Pilpa et al., 2006) is supported by the striking differences observed between what is the heme binding pocket in IsdA NEAT structure and the equivalent region in the IsdH/HarA N-terminal NEAT domain solution structure. Structural alignment of the IsdA and IsdH/HarA NEAT domain structures reveals that Tyr¹⁶⁶, which coordinates to heme-iron in IsdA, is replaced by Glu in IsdH/HarA. Tyr¹⁷⁰ is conserved; however, conformational differences in the main chain result in directing the phenol group into the heme pocket in the location of the pyrrole ring observed in the IsdA crystal structure. In the solution structure of the IsdH/HarA NEAT domain, the loop between β-strand 1b and β2 (residues Gln124 to Ser130) is disordered (Pilpa et al., 2006). The corresponding region of IsdA, consisting of residues Lys⁸¹ to Tyr⁸⁷ is highly ordered in all six molecules in the asymmetric units of the apo and holo structures (B-factors <20 Å²). This loop in IsdA interacts directly with the heme propionates (Ser⁸², His⁸³) as well as providing hydrophobic contacts to the heme (Met⁸⁴, Tyr⁸⁷). Residues 81 to 87 are not involved in crystal contact and this loop is also anchored to the hydrophic core in both the apo and holo structures by residue Met⁸⁴. Thus, for heme to bind to IsdH/HarA N-terminal NEAT domain analogous to that observed for IsdA would require substantial conformational rearrangement. Significantly, no large scale conformational change is observed between the apo and holo IsdA NEAT domain structures (FIG. 11C).

IsdA NEAT domain residues Tyr¹⁶⁶ and Tyr¹⁷⁰ are critical for heme-iron coordination and these residues are conserved in IsdC and in the C-terminal NEAT domains of IsdB and IsdH/HarA. Therefore, we anticipate that each of the four cell wall anchored S. aureus Isd proteins has at least one NEAT domain that binds heme. The sequence variation among Isd NEAT domains, including positions associated with heme binding, may serve to differentiate these proteins with respect to the recognition of unique heme-proteins and non-heme proteins. Given the differences in the ‘heme pocket’ in the N-terminal IsdH/HarA NEAT domain and the IsdA NEAT domain, coupled with observed differences in binding substrates, additional NEAT domain structures and co-crystal structures will undoubtedly shed more light upon the biological role that the individual domains play and will initiate more detailed studies on the working hypothesis that these domains are involved in transfer of heme across the envelope of Gram-positive bacteria.

TABLE 1
Data collection and refinement statistics for apo and holo IsdA NEAT domains.

| | |
|---|---|
| apo IsdA_{NEAT} | holo IsdA_{NEAT} |

Data collection*

| Resolution Range (Å) | 45-1.6 | (1.64-1.60) | 50-1.9 | (1.95-1.90) |
|---|---|---|---|---|
| Space group | P2₁ | | P2₁ | |
| Unit cell dimensions (Å) | a = 44.5, b = 58.3, | | a = 56.02, b = 58.6, | |
| | c = 45.2, β = 95.4° | | c = 96.03, β = 93.0° | |
| Unique Reflections | 28462 | | 45710 | |
| Completeness (%) | 100 | (100) | 98.3 | (96.2) |
| Average I/σI | 21.7 | (4.5) | 15.7 | (3.6) |
| Redundancy | 5.1 | (3.7) | 5.5 | (3.0) |
| R_{merge} | 0.07 | (0.26) | 0.09 | (0.29) |

Refinement

| | | | | |
|---|---|---|---|---|
| R_{work} (R_{free}) | 0.166 | (0.208) | 0.171 | (0.213) |

| | | |
|---|---|---|
| No. of water molecules | 398 | 498 |
| Average B-values (Å²) | 16.9 | 19.5 |
| r.m.s.d bond length (Å) | 0.013 | 0.013 |

Ramachandran plot, residues

| | | |
|---|---|---|
| In most-favourable region (%) | 89.6 | 90.8 |
| In disallowed regions (%) | 0.0 | 0.0 |

*Values for the highest resolution shell are shown in parenthesis

### EXAMPLE 14

### NEAT Domain Analysis: Experimental Procedures

S. aureus strains and growth conditions. S. aureus RN6390 carrying an insertionally-inactivated isdA gene has been described previously (Taylor and Heinrichs, 2002). This strain was used to generate the S. aureus Newman strain H734 via transduction using phage 80α (Sebulsky et al., 2000). Similarly, the multicopy plasmid encoding isdA (Taylor and Heinrichs, 2002), pJT35, was transduced into H734. S. aureus strains were routinely cultured at 37° C. in tryptic soy broth (Difco). Tris-minimal succinate medium (TMS) was used for iron restricted growth, prepared as previously described (Sebulsky et al., 2000). Residual free iron was chelated from TMS medium using either 2,2′ dipyridyl (200 μM) or ethylenediamine-di(o-hydroxyphenylacetic acid) (EDDHA) (10 μM). When appropriate, the antibiotics tetracycline (4 μg/ml) and chloramphenicol (5 μg/ml), were included in the growth media.

Peroxidase staining. S. aureus cell-wall extracts were prepared as previously described (Cheung and Fischetti, 1988). Briefly, cells were grown overnight in TMS broth containing 2,2′ dipyridyl. Cells were washed with 0.9% saline and incubated in cell-wall digestion buffer for 2 hrs at 37° C. Protoplasts were removed by centrifugation. The remaining cell-wall fraction was divided into two samples and either hemin (Sigma), dissolved in 0.1 N NaOH, to a final concentration of 0.1 μg/ml, or the equivalent volume of 0.1N NaOH was added to each sample. The samples were incubated for 1 hr at 37° C. Cell-wall fractions were separated by SDS-PAGE and stained with either coomassie brilliant blue R-250 (Sigma) or 3,3′5,5′ tetramethylbenzidine as previously described (Stugard et al., 1989).

Heme bioassays. S. aureus Newman strains, cultured overnight in TMS broth, were diluted into TMS broth containing 200 μM 2,2′ dipyridyl and grown to an optical density at 600 nm of 0.3-0.5. Cells were washed three times in sterile saline and added to cooled TMS agar containing EDDHA to a final concentration of 10⁵ cfu/ml. Equal volumes of the agar were poured into sterile Petri plates. Sterile paper discs saturated with hemin (10 μg) were placed on the agar plates and incubated for 72 hrs at 37° C. at which point the diameter of growth around the paper discs was recorded.

Bacterial Growth Curves. S. aureus cultures were pre-grown, from single colony, overnight in TSB. The cells were washed with saline, and 10⁷ CFU of each strain was inoculated into TMS medium containing 10 μM EDDHA with or without either 50 μM FeCl₃ or 5 μg/mL hemin. Cultures (300 μL) were incubated at 37° C. with continuous shaking and bacterial growth was monitored every 30 minutes over 20 hours using a Bioscreen C (MTX Lab Systems, Inc.). Growth curves were plotted using Sigma Plot 2000.

Recombinant protein expression. The IsdA NEAT domain coding region (residues 62-184) was cloned into pET28a (Novagen). The domain was expressed in E. Coli BL21 grown in 2× YT media (Difco) supplemented with 25 μg/ml kanamycin. Cultures were grown at 30° C. to an optical density of 0.8. Isopropyl β-D-thiogalactopyranoside (IPTG) (0.5 mM) was added and cells were incubated for 16 hours at 25° C. The His-6-tagged domain was purified using a Chelating Sepharose Fast Flow Ni²⁺ column (GE Healthcare) and dialyzed against 50 mM HEPES, pH 7.2. The His-6-tag was removed by thrombin digestion. Apo protein was isolated using a Source S column (GE Healthcare) and dialyzed against 20 mM Tris, pH 8.0. Holo protein was generated as previously described, using 20 mM Tris, pH 8 as Buffer A (Chan et al., 2006). Selenomethionine labelled protein was expressed as described previously (Van Duyne et al., 1993) and purified as above.

The portion of the isdA gene corresponding to amino acids 48-316 (omitting codons for the signal peptide and the cell wall anchoring motifs) was cloned into the GST fusion vector pGEX-2T TEV (Amersham Biosciences). GST-IsdA was expressed from E. coli ER2566 grown in Luria-Bertani broth (Difco) supplemented with 100 μg/ml ampicillin at 37° C. to an OD of 0.8. IPTG (0.4 mM) was added and cultures were grown for 20 h at room temperature. GST-IsdA was purified using a GSTPrep column (Amersham Biosciences). GST-IsdA was eluted from the column with 10 mM reduced glutathione, 100 mM NaCl, and 50 mM Tris Cl, pH 9.0 and dialyzed into phosphate buffered saline (PBS).

IsdA NEAT domain structure determination. Crystals were grown at 19° C. by hanging drop vapour diffusion. Drops contained 1 μl of 25 mg/ml protein and 1 μl of reservoir solution. The apo-protein reservoir contained 0.1 M CHES, pH 9.5, 30% PEG 4000. Crystals were transferred to mother liquor supplemented to 15% glycerol and flash frozen in liquid nitrogen. The reservoir for holo-protein contained 0.1 M MES, pH 6.5, 0.2 M ammonium sulphate, 30% PEG 6000. Crystals were transferred into mother liquor supplemented to 20% glycerol and immersed in liquid nitrogen. Heme content of holo crystals was examined by dissolving crystals in water and analyzing them by electronic spectroscopy using a Varian Cary Bio50 UV spectrophotometer and an 80 μl quartz cuvettes with a 1 cm path length (FIG. S2).

X-ray diffaction data were collected at the Stanford Synchrotron Radiation Laboratory (Menlo Park, Calif.) at 100 K on beamline 1-5. Multiple wavelength anomalous diffraction data was collected for SeMet labelled apo-protein at the selenium peak (0.97879 Å) and inflection (0.97927 Å) wavelengths. Holo IsdA NEAT domain data sets were collected at a wavelength of 0.97944 Å. Data was processed and scaled with HKL2000 (Otwinowski and Minor, 1997). Apo and holo IsdA NEAT domain crystals are not isomorphous and had two and four molecules in the asymmetric unit, respectively. Initial phases and a preliminary model of the apo structure were determined using Solve (Terwilliger and Berendzen, 1999) and Resolve (Terwilliger, 2000, 2003). Manual construction of the molecule was done using the program O (Jones et al., 1991) and refined with Refmac5 (Murshudov et al., 1997) from the CCP4 program suite (Collaborative Computational Project, 1994). Molecular replacement with the apo form as a search model was used to solve the holo structure using the program Molrep from CCP4 (Collaborative Computational Project, 1994) and refined as above. Crystallographic data and refinement statistics are shown in Table 1.

For the apo structure, chain A was used for figures and structural comparisons since chain B contained a CHES buffer molecule bound in the heme pocket altering the conformation of neighbouring residues. Chain A from the holo structure was used for figures and analysis since chains C and D form a crystal contact at their respective heme binding pockets. Chain A and B molecular contacts are removed from the heme binding site. See FIGS. S3 and S4. Figures were generated in PYMOL (DeLano Scientific, San Carlos, Calif.).

Heme binding by native and mutant forms of IsdA. Site-directed mutagenesis of isdA was performed using the QuikChange® PCR Kit (Invitrogen), with Pfu Turbo® polymerase and pGST-IsdA as a template. The PCR products were immediately DpnI (Roche) treated for 45 min to degrade template DNA, and transformed into E. coli ER2566. Mutations were confirmed by sequencing at the Robarts Research Institute DNA Sequencing Facility (London, Ontario).

GST-IsdA proteins were purified as described above. Wild type and point mutant proteins were purified as expressed from E. coli and relative heme binding was assessed based upon the ability of the proteins, all expressed in an equivalent fashion, to scavenge and retain association with heme derived from the cytoplasm of E. coli Proteins were adjusted to an equivalent concentration and electronic spectra were recorded using a Cary 500 spectrophotometer (Varian Inc.) with a 1 cm path length and 1 mL quartz cuvettes. All recordings were taken at room temperature.

### REFERENCES

Ahn, S. H., Han, J. H., Lee, J. H., Park, K. J., and Kong, I. S. (2005) Identification of an iron-regulated hemin-binding outer membrane protein, HupO, in Vibrio fluvialis: effects on hemolytic activity and the oxidative stress response. Infect Immun 73: 722-729.
Andrade, M. A., Ciccarelli, F. D., Perez-Iratxeta, C., and Bork, P. (2002) NEAT: a domain duplicated in genes near the components of a putative Fe³⁺ siderophore transporter from Gram-positive pathogenic bacteria. Genome Biol 3: RESEARCH0047.
Amoux, P., Haser, R., Izadi, N., Lecroisey, A., Delepierre, M., Wandersman, C., and Czjzek, M. (1999) The crystal structure of HasA, a hemophore secreted by Serratia marcescens. Nat Struct Biol 6: 516-520.
Chan, A. C., Lelj-Garolla, B., Rosell, F. I., Pedersen, K. A., Mauk, A. G., and Murphy, M. E. (2006) Cofacial heme binding is linked to dimerization by a bacterial heme transport protein. J Mol Biol 362: 1108-1119.
Cheung, A. L., and Fischetti, V. A. (1988) Variation in the expression of cell wall proteins of Staphylococcus aureus grown on solid and liquid media. Infect Immun 56: 1061-1065.
Clarke, S. R., Wiltshire, M. D., and Foster, S. J. (2004) IsdA of Staphylococcus aureus is a broad spectrum iron-regulated adhesin. Mol Microbiol 51: 1509-1519.
Clarke, S. R., Brummell, K. J., Horsburgh, M. J., McDowell, P. W., Mohamad, S. A., Stapleton, M. R., et al. (2006) Identification of in vivo-expressed antigens of Staphylococcus aureus and their use in vaccinations for protection against nasal carriage. J Infect Dis 193: 1098-1108.
Dryla, A., Gelbmann, D., von Gabain, A., and Nagy, E. (2003) Identification of a novel iron regulated staphylococcal surface protein with haptoglobin-haemoglobin binding activity. Mol Microbiol 49: 37-53.
Eakanunkul, S., Lukat-Rodgers, G. S., Sumithran, S., Ghosh, A., Rodgers, K. R., Dawson, J. H., and Wilks, A. (2005) Characterization of the periplasmic heme-binding protein shut from the heme uptake system of Shigella dysenteriae. Biochemistry 44: 13179-13191.
Hall, T. (1999) BioEdit: a user-friendly biological sequence alignment editor and analysis program for Windows 95/98/NT. Nucleic Acids Symp Ser 41: 95-98.
Hendrickson, W. A. (1991) Determination of macromolecular structures from anomalous diffraction of synchrotron radiation. Science 254: 51-58.
Holm L., and Sander, C. (1993) Protein structure comparison by alignment of distance matrices. J Mol Biol 233: 123-138.
Izadi, N., Henry, Y., Haladjian, J., Goldberg, M. E., Wandersman, C., Delepierre, M., and Lecroisey, A. (1997) Purification and characterization of an extracellular heme-binding protein, HasA, involved in heme iron acquisition. Biochemistry 36: 7050-7057.
Jones, T. A., Zou, J. Y., Cowan, S. W., and Kjeldgaard (1991) Improved methods for building protein models in electron density maps and the location of errors in these models. Acta Crystallogr A 47 (Pt 2): 110-119.
Kuklin, N. A., Clark, D. J., Secore, S., Cook, J., Cope, L. D., McNeely, T., et al. (2006) A novel Staphylococcus aureus vaccine: iron surface determinant B induces rapid antibody responses in rhesus macaques and specific increased survival in a murine S. aureus sepsis model. Infect Immun 74: 2215-2223.
Mack J., Vermeiren, C., Heinrichs, D. E., and Stillman, M. J. (2004) In vivo heme scavenging by Staphylococcus aureus IsdC and IsdE proteins. Biochem Biophys Res Commun 320:781-788.
Marraffini, L. A., Dedent, A. C., and Schneewind, O. (2006) Sortases and the art of anchoring proteins to the envelopes of gram-positive bacteria. Microbiol Mol Biol Rev 70: 192-221.
Mauk, A., and Moore, G. (1997) Control of metalloprotein redox potentials: what does site-directed mutagenesis of hemoproteins tell us? J Biol Inorg Chem 2: 119-125.
Mazmanian, S. K., Ton-That, H., Su, K., and Schneewind, O. (2002) An iron-regulated sortase anchors a class of surface protein during Staphylococcus aureus pathogenesis. Proc Natl Acad Sci USA 99: 2293-2298.
Mazmanian, S. K., Skaar, E. P., Gaspar, A. H., Humayun, M., Gornicki, P., Jelenska, J., et al. (2003) Passage of heme-iron across the envelope of Staphylococcus aureus. Science 299: 906-909.
Murphy, E. R., Sacco, R. E., Dickenson, A., Metzger, D. J., Hu, Y., Orndorff, P. E., and Connell, T. D. (2002) BhuR, a virulence-associated outer membrane protein of Bordetella avium, is required for the acquisition of iron from heme and hemoproteins. Infect Immun 70: 5390-5403.
Murshudov, G. N., Vagin, A. A., and Dodson, E. J. (1997) Refinement of macromolecular structures by the maximum-likelihood method. Acta Crystallogr D Biol Crystallogr 53: 240-255.
Otwinowski, Z., and Minor, W. (1997) Processing of x-ray diffraction data collected in oscillation mode. Methods Enzymol 276: 307-326.
Paoli, M., Anderson, B. F., Baker, H. M., Morgan, W. T., Smith, A., and Baker, E. N. (1999) Crystal structure of hemopexin reveals a novel high-affinity heme site formed between two beta-propeller domains. Nat Struct Biol 6: 926-931.
Pilpa, R. M., Fadeev, E. A., Villareal, V. A., Wong, M. L., Phillips, M., and Clubb, R. T. (2006) Solution structure of the NEAT (NEAr Transporter) domain from IsdH/HarA: the human hemoglobin receptor in Staphylococcus aureus. J Mol Biol 360: 435-437.
Putnam, C. D., Arvai, A. S., Bourne, Y., and Tainer, J. A. (2000) Active and inhibited human catalase structures: ligand and NADPH binding and catalytic mechanism. J Mol Biol 296: 295-309.
Ratledge, C., and Dover, L. G. (2000) Iron metabolism in pathogenic bacteria. Annu Rev Microbiol 54: 881-941.
Schneider, S., Sharp, K. H., Barker, P. D., and Paoli, M. (2006) An induced fit conformational change underlies the binding mechanism of the heme-transport proteobacteria-protein HemS. J Biol Chem. 281: 32606-32610.
Sebulsky, M. T., Hohnstein, D., Hunter, M. D., and Heinrichs, D. E. (2000) Identification and characterization of a membrane permease involved in iron-hydroxamate transport in Staphylococcus aureus. J Bacteriol 182: 4394-4400.
Skaar, E. P., Humayun, M., Bae, T., DeBord, K. L., and Schneewind, O. (2004) Iron-source preference of Staphylococcus aureus infections. Science 305: 1626-1628.
Skaar, E. P., and Schneewind, O. (2004) Iron-regulated surface determinants (Isd) of Staphylococcus aureus: stealing iron from heme. Microbes Infect 6: 390-397.
Stojiljkovic, I., Hwa, V., de Saint Martin, L., O'Gaora, P., Nassif, X., Heffron, F., and So, M. (1995) The Neisseria meningitidis haemoglobin receptor: its role in iron utilization and virulence. Mol Microbiol 15: 531-541.
Stojiljkovic, I., and Perkins-Balding, D. (2002) Processing of heme and heme-containing proteins by bacteria. DNA Cell Biol 21: 281-295.
Stugard, C. E., Daskaleros, P. A., and Payne, S. M. (1989) A 101-kilodalton heme-binding protein associated with congo red binding and virulence of Shigella flexneri and enteroinvasive Escherichia coli strains. Infect Immun 57: 3534-3539.
Taylor, J. M., and Heinrichs, D. E. (2002) Transferrin binding in Staphylococcus aureus: involvement of a cell wall-anchored protein. Mol Microbiol 43: 1603-1614.
Terwilliger, T. C., and Berendzen, J. (1999) Automated MAD and MIR structure solution. Acta Crystallogr D Biol Crystallogr 55: 849-861.
Terwilliger, T. C. (2000) Maximum-likelihood density modification Acta Crystallogr D Biol Crystallogr 56: 965-972.
Terwilliger, T. C. (2003) Automated main-chain model building by template matching and iterative fragment extension. Acta Crystallogr D Biol Crystallogr 59: 38-44.
Uchida, T., Stevens, J. M., Daltrop, O., Harvat, E. M., Hong, L., Ferguson, S. J., and Kitagawa, T. (2004) The interaction of covalently bound heme with the cytochrome c maturation protein CcmE. J Biol Chem 279: 51981-51988.
Van Duyne, G. D., Standaert, R. F., Karplus, P. A., Schreiber, S. L., and Clardy, J. (1993) Atomic structures of the human immunophilin FKBP-12 complexes with FK506 and rapamycin. J Mol Biol 229: 105-124.
Vermeiren, C. L., Pluym, M., Mack, J., Heinrichs, D. E., and Stillman, M. J. (2006) Characterization of the heme binding properties of Staphylococcus aureus IsdA. Biochemistry 45: 12867-12875.
Wandersman, C., and Delepelaire, P. (2004) Bacterial iron sources: from siderophores to hemophores. Annu Rev Microbiol 58: 611-647.
Zunszain, P. A., Ghuman, J., Komatsu, T., Tsuchida, E., and Curry, S. (2003) Crystal structural analysis of human serum albumin complexed with hemin and fatty acid. BMC Struct Biol 3:6.

The practice of the present invention may employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning: A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Antibodies: A Laboratory Manual, and Animal Cell Culture (R. I. Freshney, ed. (1987)), Manipulating the Mouse Embryo, (Cold Spring Harbor Laboratory Press, Cold Spring Harbor, N.Y., 1986).

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

Equivalents

Those skilled in the art will recognize, or be able to ascertain using no more than routine experimentation, many equivalents to the specific embodiments of the invention described herein. Such equivalents are intended to be encompassed by the following claims.

1. A pharmaceutical composition selected from the group consisting of:
(a) a vaccine comprising an Isd NEAT domain polypeptide and a pharmaceutically acceptable carrier;
(b) a pharmaceutical composition comprising an effective anti-bacterial amount of an antibody that binds to an Isd NEAT domain polypeptide and a pharmaceutically acceptable carrier; and
(c) a pharmaceutical composition comprising a nucleic acid that is antisense to a nucleic acid encoding an Isd NEAT domain and a pharmaceutically acceptable carrier.

2. The pharmaceutical composition of claim 1, which is a vaccine, and wherein the vaccine is within an injectable formulation.

3. The pharmaceutical composition of claim 1, which further comprises an adjuvant.

4. (canceled)

5. (canceled)

6. A method for identifying an agent that binds to a Isd NEAT domain and inhibits the uptake of iron comprising,
(i) contacting the Isd NEAT domain with an appropriate interacting molecule in the presence of an agent under conditions permitting the interaction between the Isd NEAT domain and the interacting molecule in the absence of an agent; and
(ii) determining the level of interaction between the Isd NEAT domain and the interacting molecule, wherein a different level of interaction between the Isd NEAT domain and the interacting molecule in the presence of the agent relative to the absence of the agent indicate that the agent inhibits the interaction between the Isd NEAT domain and the interacting molecule.

7. The method of claim 6, wherein the Isd NEAT domain is selected from the group consisting of a NEAT domain of Staphylococcus aureus IsdA, IsdB, and IsdC.

8. A method for identifying an agent that inhibits the expression of a Staphylococcus aureus Isd NEAT domain sequence comprising:
(i) culturing a wild type Staphylococcus aureus strain in the presence or absence of said agent; and
(ii) comparing the expression of an Isd sequence wherein a greater reduction in the expression of an Isd sequence in cells treated with said agent indicates that said agent inhibits the expression of an Isd sequence in Staphylococcus aureus.

9. The method of claim 8, wherein the Isd NEAT domain sequence is a nucleic acid sequence.

10. The method of claim 8, wherein the Isd NEAT domain sequence is a polypeptide sequence.


SEQUENCE LISTING (19 sequences)

SEQ ID NO: 1 - DNA, 1053 bp, Staphylococcus aureus
atgacaaaacattatttaaacagtaagtatcaatcagaacaacgttcatcagctatgaaaaagattacaatgggtacagcatctatcattttaggttcccttgtatacataggcgcagacagccaacaagtcaatgcggcaacagaagctacgaacgcaactaataatcaaagcacacaagtttctcaagcaacatcacaaccaattaatttccaagtgcaaaaagatggctcttcagagaagtcacacatggatgactatatgcaacaccctggtaaagtaattaaacaaaataataaatattatttccaaaccgtgttaaacaatgcatcattctggaaagaatacaaattttacaatgcaaacaatcaagaattagcaacaactgttgttaacgataataaaaaagcggatactagaacaatcaatgttgcagttgaacctggatataagagcttaactactaaagtacatattgtcgtgccacaaattaattacaatcatagatatactacgcatttggaatttgaaaaagcaattcctacattagctgacgcagcaaaaccaaacaatgttaaaccggttcaaccaaaaccagctcaacctaaaacacctactgagcaaactaaaccagttcaacctaaagttgaaaaagttaaacctactgtaactacaacaagcaaagttgaagacaatcactctactaaagttgtaagtactgacacaacaaaagatcaaactaaaacacaaactgctcatacagttaaaacagcacaaactgctcaagaacaaaataaagttcaaacacctgttaaagatgttgcaacagcgaaatctgaaagcaacaatcaagctgtaagtgataataaatcacaacaaactaacaaagttacaaaacataacgaaacgcctaaacaagcatctaaagctaaagaattaccaaaaactggtttaacttcagttgataactttattagcacagttgccttcgcaacacttgcccttttaggttcattatctttattacttttcaaaagaaaagaatctaaataa

SEQ ID NO: 2 - DNA, 1053 bp, Staphylococcus aureus
ttatttagattcttttcttttgaaaagtaataaagataatgaacctaaaagggcaagtgttgcgaaggcaactgtgctaataaagttatcaactgaagttaaaccagtttttggtaattctttagctttagatgcttgtttaggcgtttcgttatgttttgtaactttgttagtttgttgtgatttattatcacttacagcttgattgttgctttcagatttcgctgttgcaacatctttaacaggtgtttgaactttattttgttcttgagcagtttgtgctgttttaactgtatgagcagtttgtgttttagtttgatcttttgttgtgtcagtacttacaactttagtagagtgattgtcttcaactttgcttgttgtagttacagtaggtttaactttttcaactttaggttgaactggtttagtttgctcagtaggtgttttaggttgagctggttttggttgaaccggtttaacattgtttggttttgctgcgtcagctaatgtaggaattgctttttcaaattccaaatgcgtagtatatctatgattgtaattaatttgtggcacgacaatatgtactttagtagttaagctcttatatccaggttcaactgcaacattgattgttctagtatccgcttttttattatcgttaacaacagttgttgctaattcttgattgtttgcattgtaaaatttgtattctttccagaatgatgcattgtttaacacggtttggaaataatatttattattttgtttaattactttaccagggtgttgcatatagtcatccatgtgtgacttctctgaagagccatctttttgcacttggaaattaattggttgtgatgttgcttgagaaacttgtgtgctttgattattagttgcgttcgtagcttctgttgccgcattgacttgttggctgtctgcgcctatgtatacaagggaacctaaaatgatagatgctgtacccattgtaatctttttcatagctgatgaacgttgttctgattgatacttactgtttaaataatgttttgtcat

SEQ ID NO: 3 - protein, 350 aa, Staphylococcus aureus
MTKHYLNSKYQSEQRSSAMKKITMGTASIILGSLVYIGADSQQVNAATEATNATNNQSTQVSQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLADAAKPNNVKPVQPKPAQPKTPTEQTKPVQPKVEKVKPTVTTTSKVEDNHSTKVVSTDTTKDQTKTQTAHTVKTAQTAQEQNKVQTPVKDVATAKSESNNQAVSDNKSQQTNKVTKHNETPKQASKAKELPKTGLTSVDNFISTVAFATLALLGSLSLLLFKRKESK

SEQ ID NO: 4 - DNA, 1938 bp, Staphylococcus aureus
atgaacaaacagcaaaaagaatttaaatcattttattcaattagaaagtcatcactaggcgttgcatctgtagcgattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagcagttaaaccagcagcaaaagccactaacaatacatatcctattttgaatcaggaacttagagaagcgattaaaaaccctgcaataaaagataaagatcatagcgcaccaaactctcgtccaattgattttgaaatgaaaaaagaaaatggtgagcaacaattttatcattatgccagctctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattacgcttacattcgcttctctgtttcaaatggaacaaaagccgttaaaattgtaagttcaactcacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattactagctttaagtagcatcgttgcattcgtattacctagaaaacgtaaaaactaa

SEQ ID NO: 5 - DNA, 1938 bp, Staphylococcus aureus
ttagtttttacgttttctaggtaatacgaatgcaacgatgctacttaaagctagtaatgccattaatggtaatgtcatatctttatttgattcttcaccagtttgtggtaatgattttgctttattttcttgtgtatttttattgttttggctttgagtgtgtccatcatttgtgtttttaatgtttgctttttgtaatggagcactatcttttgcttcgctagaacctgctgaagtttgaacaacatcttttgttgtttttgatgaagcagttgttggttttgcaacattttgagtcgtagatactaccttagttggagttgtactacttgattctacttcacctttagttggttttgtagcaggcgttttgtctttacctgactcactagatgcgtcattttctttttcaacacttggtaattgtttattgtcatctttttggctgtcttgtttttgtgattctttttcaacaggtgatggtgttggtttgctaggcgtagctggagtagcttccttcttagctgagttatcttgttgttcttttttgttagatttatcggtattggcttttgtaaatgcttctttatcaacgattctgacatggtattgtccatcataatcaatcgtttttacgtgaactttaacgatagcatcatatagagttttaccttcaacatatgggaaaataattgttctagtattatttttagcatctttgcttatagttctaacacgttgaccttcaaccatgaaatctttccagtaatcgtcattagtagtttccatgaccatatattttttgccgttaagcatacctgttttaatagggtgtttaacaaaagtatccatcatagattcgttattctcaacactttcataaacaacatattttgtatcttgtaaatcagtcattttttcatttgttggttgtacattttggaattcagtaatagctgatttcacttgctcatctaaagctttctttgtatcctctaatttcttcttgtactcagcctttaatttttcaggaagtttatcttgaattttatttaattcataaacttgtctttctagtgttttcgcttttttatatggcgctaataatttttcagctttataatcttcttcagttttgaatttatctgcactgttataaattggttgtgcgaattccattaatgtgtaatcgtatttttcttctttgttattgaagtgagttgaacttacaattttaacggcttttgttccatttgaaacagagaagcgaatgtaagcgtaatctttaacagtatcgtatgatactaatttaattggcaactttttgtcaccttcataaacttcaaattttctccaaaattgacctgattgtaatcctaattcaatttctggttttgaatcagtgaaaataactctagcaggtttaacagagctggcataatgataaaattgttgctcaccattttcttttttcatttcaaaatcaattggacgagagtttggtgcgctatgatctttatcttttattgcagggtttttaatcgcttctctaagttcctgattcaaaataggatatgtattgttagtggcttttgctgctggtttaactgcttttgtttccttaggggctttaacttctttaacttctttagcttcttttgtttcagaagtaggggcctcaacttctttattagatactgagacagcattagctactggtttagtttctggagctttttcagatgttgttgttggacttgcaactgcttcagtttttggttgtgcttctgtatttgtaccacctgtttcttcagctgctgcttgtgcttcgccatttgacattaataataaaagtgtactaatcgctacagatgcaacgcctagtgatgactttctaattgaataaaatgatttaaattctttttgctgtttgttcat

SEQ ID NO: 6 - protein, 645 aa, Staphylococcus aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 7 - DNA, 684 bp, Staphylococcus aureus
atgaaaaatattttaaaagtttttaatacaacgattttagcgttaattatcatcatcgcgacattcagtaattctgcaaatgccgcagatagcggtactttgaattatgaggtttacaaatacaataccaatgacacgtcaattgctaatgactattttaataaaccggcaaagtacattaagaaaaatggtaaattgtatgttcaaataactgtcaaccacagtcattggattactggaatgagtatcgaaggacataaagaaaatattattagtaaaaacactgccaaagatgaacgcacttctgaatttgaagtaagtaagttgaacggtaaaatagatggaaaaattgacgtttatatcgatgaaaaagtaaatggaaagccattcaaatatgaccatcattacaacattacatataaatttaatggaccaactgatgtagcaggtgctaatgcaccaggtaaagatgataaaaattctgcttcaggtagtgacaaaggatctgatggaacgactactggtcaaagtgaatctaatagttcgaataaagacaaagtagaaaatccacaaacaaatgctggtacacctgcatatatatatgcaataccagttgcatccttagcattattaatcgcaatcacattgtttgttagaaaaaaatctaaaggcaatgtggaataa

SEQ ID NO: 8 - DNA, 684 bp, Staphylococcus aureus
ttattccacattgcctttagatttttttctaacaaacaatgtgattgcgattaataatgctaaggatgcaactggtattgcatatatatatgcaggtgtaccagcatttgtttgtggattttctactttgtctttattcgaactattagattcactttgaccagtagtcgttccatcagatcctttgtcactacctgaagcagaatttttatcatctttacctggtgcattagcacctgctacatcagttggtccattaaatttatatgtaatgttgtaatgatggtcatatttgaatggctttccatttactttttcatcgatataaacgtcaatttttccatctattttaccgttcaacttacttacttcaaattcagaagtgcgttcatctttggcagtgtttttactaataatattttctttatgtccttcgatactcattccagtaatccaatgactgtggttgacagttatttgaacatacaatttaccatttttcttaatgtactttgccggtttattaaaatagtcattagcaattgacgtgtcattggtattgtatttgtaaacctcataattcaaagtaccgctatctgcggcatttgcagaattactgaatgtcgcgatgatgataattaacgctaaaatcgttgtattaaaaacttttaaaatatttttcat

SEQ ID NO: 9 - protein, 227 aa, Staphylococcus aureus
MKNILKVFNTTILALIIIIATFSNSANAADSGTLNYEVYKYNTNDTSIANDYFNKPAKYIKKNGKLYVQITVNHSHWITGMSIEGHKENIISKNTAKDERTSEFEVSKLNGKIDGKIDVYIDEKVNGKPFKYDHHYNITYKFNGPTDVAGANAPGKDDKNSASGSDKGSDGTTTGQSESNSSNKDKVENPQTNAGTPAYIYAIPVASLALLIAITLFVRKKSKGNVE

SEQ ID NO: 10 - protein, 5 aa, Artificial Sequence
  Description of Artificial Sequence Synthetic peptide motif
  MOD_RES (3): variable amino acid residue
LPXTG

SEQ ID NO: 11 - protein, 5 aa, Artificial Sequence
  Description of Artificial Sequence Synthetic peptide motif
NPQTN

SEQ ID NO: 12 - protein, 4 aa, Artificial Sequence
  Description of Artificial Sequence Illustrative ER retention Signal peptide
KDEL

SEQ ID NO: 13 - protein, 123 aa, Staphylococcus aureus
SQATSQPINFQVQKDGSSEKSHMDDYMQHPGKVIKQNNKYYFQTVLNNASFWKEYKFYNANNQELATTVVNDNKKADTRTINVAVEPGYKSLTTKVHIVVPQINYNHRYTTHLEFEKAIPTLA

SEQ ID NO: 14 - protein, 121 aa, Staphylococcus aureus
SAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSA

SEQ ID NO: 15 - protein, 119 aa, Staphylococcus aureus
KMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANT

SEQ ID NO: 16 - protein, 125 aa, Staphylococcus aureus
NAADSGTLNYEVYKYNTNDTSIANDYFNKPAKYIKKNGKLYVQITVNHSHWITGMSIEGHKENIISKNTAKDERTSEFEVSKLNGKIDGKIDVYIDEKVNGKPFKYDHHYNITYKFNGPTDVAGA

SEQ ID NO: 17 - protein, 123 aa, Staphylococcus aureus
DIGPREQVNFQLLDKNNETQYYHFFSIKDPADVYYTKKKAEVELDINTASTWKKFEVYENNQKLPVRLVSYSPVPEDHAYIRFPVSDGTQELKIVSSTQIDDGEETNYDYTKLVFAKPIYNDP

SEQ ID NO: 18 - protein, 122 aa, Staphylococcus aureus
TADNWRPIDFQMKNDKGERQFYHYASTVEPATVIFTKTGPVIELGLKTASTWKKFEVYEGDKKLPVELVSYDSDKDYAYIRFPVSNGTRDVKNVSSIEYGENIHEDYDYTLMVFAQPITNNP

SEQ ID NO: 19 - protein, 119 aa, Staphylococcus aureus
QLTDLQEAHFVVFESEENSESVMDGFVEHPFYTATLNGQKYVVMKTKDDSYWKDLIVEGKRVTTVSKDPKNNSRTLIFPYIPDKAVYNAIVKVVVANIGYEGQYHVRIINQDINTKDDD